(19) United States
(12) Patent Application Publication
(10) Pub. No.: US 2015/0157672 A1
(43) Pub. Date: Jun. 11, 2015

(54) KITS AND METHODS FOR SUSTAINED WEIGHT LOSS

(71) Applicant: PHYTOLOGY LABS, INC., New York, NY (US)

(72) Inventor: Brian CAIRNS, New York, NY (US)

(21) Appl. No.: 14/563,764

(22) Filed: Dec. 8, 2014

Related U.S. Application Data
(60) Provisional application No. 61/913,459, filed on Dec. 9, 2013.

(51) Int. Cl. A61K 36/03; A61K 36/82; A61K 36/87; A61K 36/77; A61K 31/475; A61K 36/53; A61K 36/73; A61K 36/752; A61K 36/38; A61K 36/48; A61K 36/185

(52) U.S. Cl. CPC A61K 36/03; A61K 36/48; A61K 36/82; A61K 36/87; A61K 36/77; A61K 36/185; A61K 36/53; A61K 36/73; A61K 36/752; A61K 36/38; A61K 31/475

(57) ABSTRACT

Disclosed herein are compositions, methods and kits of effecting and prolonging weight loss by administering weight loss agents for treatment phases of predetermined durations.

### CROSS-REFERENCE TO RELATED APPLICATIONS

This application claims priority to U.S. Provisional Application No. 61/913,459, filed Dec. 9, 2013 and entitled “KITS AND METHODS FOR SUSTAINED WEIGHT LOSS,” the contents of which are incorporated herein in their entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

Not applicable

### BRIEF SUMMARY OF THE INVENTION

Embodiments herein may be directed to a method of effecting weight loss in a subject. The method may comprise defining at least two treatment phases and administering at least one weight loss agent during each treatment phase. Each of the at least two treatment phases may have a pre-determined duration. At least one weight loss agent administered during each treatment phase may be selected from a different therapeutic class than the at least one weight loss agent administered during the preceding or proceeding treatment phase.

Some embodiments may be directed to a method of prolonging the effect of a weight loss agent in a subject. The method may include defining at least two treatment phases and administering at least one weight loss agent during each treatment phase. Each treatment phase may have a pre-determined duration. At least one weight loss agent administered during each treatment phase may be selected from a different therapeutic class than at least one weight loss agent administered during the preceding or proceeding treatment phase.

Some embodiments may be directed to a kit for effecting weight loss in a subject. The kit may include at least two weight loss agents and instructions. The instructions may be for defining at least two treatment phases and for administering at least one weight loss agent during each treatment phase. Each of the at least two treatment phases may be for a pre-determined duration. At least one weight loss agent administered during each treatment phase may be selected from a different therapeutic class than at least one weight loss agent administered during the preceding or proceeding treatment phase.

In some embodiments, a therapeutic class may be selected from a neurotransmitter modulator, a hormonal satiety or hunger signaling modulator, a β-adrenergic receptor modulator, an α2-adrenergic modulator, an adenosine receptor modulator, a cannabinoid receptor modulator, a protein-tyrosine phosphatase 1B modulator, a catechol O-methyltransferase modulator, an AMPk modulator, a phosphodiesterase modulator, a non-guanosine nucleotide mediated adenylate cyclase modulator, which may also be known as a non-G-protein adenylate cyclase modulator, a glucose metabolism modulator, a carbohydrate metabolism modulator, a lipid metabolism modulator, a protein kinase A modulator, a macro nutrient absorption modulator and an agent with an unknown mechanism of action.

In some embodiments, a neurotransmitter modulator may be selected from Griffonia simplicifolia, Crocus sativus, Mucuna pruriens, L-theanine, St. John's Wort, Catha edulis, Rhodiola rosea, Quercitin, and Garcina spp. In some embodiments, a hormonal satiety or hunger signaling modulator may be selected from Slendesta™, Fenugreek (Trigonella foenum-graecum), Plantago ovata (Plantago psyllium), Fibersol® 2, alginate, conjugated linoleic acid, α-lipoic acid, diacylglycerol, linolenate, Cocos nucifera oleum, whey protein, soy protein, Coix lacryma-jobi, Ilex paraguariensis, Quercitin, Agave tequilana, Dasylirion spp., and Panax ginseng. In some embodiments, a β-adrenergic receptor modulator may be selected from Citrus aurantium and Capsicum spp., which may also be known as a capsinoid. In some embodiments, a α₂-adrenergic receptor modulator may be selected from yohimbine and Rubus idaeus. In some embodiments, an adenosine receptor modulator may be selected from caffeine, Guarana spp., Ilex paraguariensis, xanthine alkaloids, and theobromine. In some embodiments, a cannabinoid receptor modulator may be selected from PhosphoLean®. In some embodiments, a protein-tyrosine phosphatase 1B (PTP1B) modulator may be selected from Cinnamomum cassia. In some embodiments, a catechol O-methyltransferase (COMT) modulator may be selected from Camellia sinensis and PhosphoLean®. In some embodiments, a 5′ AMP-activated protein kinase (AMPk) modulator may be selected from Salix alba, Coffea canefora, Ilex paraguariensis, Ecklonia cava, phillyrin, Rubus idaeus, Panax ginseng berry and Panax ginseng root. In some embodiments, a phosphodiesterase modulator may be selected from Sinetrol®, phillyrin, caffeine, Guarana spp., Ilex paraguariensis and theobromine. In some embodiments, a non-guanosine nucleotide mediated adenylate cyclase modulator, which may also be known as a non-G-protein adenylate cyclase modulator, may be selected from Coleus forskholii. In some embodiments, a glucose metabolism modulator may be selected from Momordica charantia, Olea europea and Theobroma cacao bean. In some embodiments, a carbohydrate metabolism modulator may be selected from Garcinia spp., Coffea canefora, Phaseolus vulgaris, Cissus quadrangularis, Irvingia gabonensis, Carthamus tinctorius, Laminaria japonica, Curcuma longa, Zingiber officinale, Rosemarinus officinalis and Gymnema sylvestre. In some embodiments, a lipid metabolism modulator are selected from an isohumulone, which may also be known as Humulus lupulus, Arachis hypogaea, Allium victorialis, Punica granatum leaf, Kochis scoparia, Panax japonicas, Aesculus turbinate, Ceratonia siliqua pulp, oolong tea, Ilex paraguariensis, Glycyrrhiza glabra, and Cissus quadrangularis. In some embodiments, an agent with an unknown mechanism of action may be selected from Caralluma fimbriata, Hoodia gordonii, Parasitic loranthus, Paeonia suffruticosa, Turnera diffusa, Sambuca nigra, Asparagus officinalis, 5-hydroxytryptophan, Slendesta®, Olea europea and acetic acid (vinegar).

In some embodiments, a predetermined treatment phase may have a pre-determined duration of about 1 week to about 20 weeks. In some embodiments, the predetermined treatment phase may have a pre-determined duration of about 4 weeks.

In some embodiments, at least two weight loss agents may be administered during each treatment phase. At least one weight loss agent administered during each treatment phase may be selected from a different therapeutic class than at least one weight loss agent administered during the preceding or proceeding treatment phase.

### DESCRIPTION OF DRAWINGS

FIG. 1 depicts a line graph showing the mean percentage weight loss from baseline to week 108 for Qysmia® (phentermine and topiramate extended-release capsules). The mean weight loss as a percent is plotted against the time in weeks. A standardized lifestyle intervention was used across all treatment groups. P<0.0001 compared with placebo (diamonds) at all time points assessed.

FIG. 2 depicts a bar graph showing the percent of total weight lost in 4-week intervals. Five weight loss agents were analyzed: calcium plus vitamin D3, alginate, green tea, Coleus forskholii and phentermine. Each bar within a particular weight loss agent represents weeks 1 through 4, weeks 5 through 8 and weeks 9 through 12, respectively.

FIG. 3 depicts a line graph showing the mean percentage change in initial basal metabolic rate (BMI) for adolescents treated with sibutramine or placebo, where P<0.05 between groups at each point (by ANOVA).

FIG. 4 depicts a line graph showing changes from the baseline to weeks 2, 4, 6, 8, 10 and 12 in body weight of overweight or obese adults who consumed a control or catechin-containing beverage. The values are least squares means+/−SEM, n=65 (catechin) and 63 (control). The values were adjusted for baseline value, age and sex. The values to the right of week 12 data are least squares means (95% CI).

FIG. 5 depicts a line graph showing pooled weight loss data from green tea supplementation over time.

FIG. 6 depicts a scatter graph showing a comparison of actual Garcina clinical weight loss studies and predicted weight loss by dosage.

FIG. 7 depicts a line graph showing results from Orlistat clinical trials, which indicates actual data versus model data.

FIG. 8 depicts a scatter graph showing results from Qysmia® clinical trials, which indicates actual data versus model data.

FIG. 9 depicts a bar graph of an average weight loss per week over three phases.

FIG. 10 depicts a scatter graph of an actual average weight loss over time.

FIG. 11 depicts a scatter graph of an actual body mass index (BMI) over time.

### DETAILED DESCRIPTION

Given its fundamental role in human survival, the body has developed a number of different pathways (referred herein as therapeutic classes) and positive/negative feedback systems to regulate weight, energy expenditure/storage, and/or the like, all of which are highly conserved. Desensitization, an attenuated response to chronic stimulation, is commonly observed in biological systems. Photoadaptation, as well as tolerance to therapeutic agents, such as insulin and catecholamines, are important examples of desensitization. As a result, weight loss products may lose effectiveness over time as the body compensates to balance out the product's effects. Such a loss of effectiveness may begin almost immediately and may plateau at about 26 weeks. A weight loss plateau may be defined as the time point when a weight loss agent ceases to be effective. A similar effect may be seen with fluoxetine, a number of serotonin uptake inhibitors (SSRIs), as well as a number of herbal preparations.

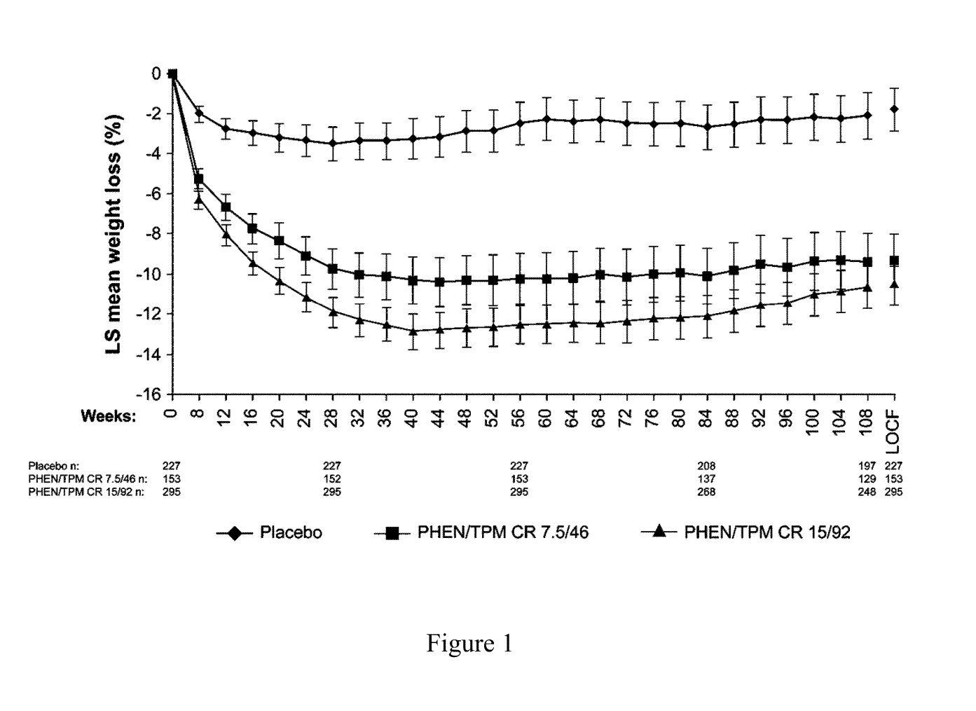

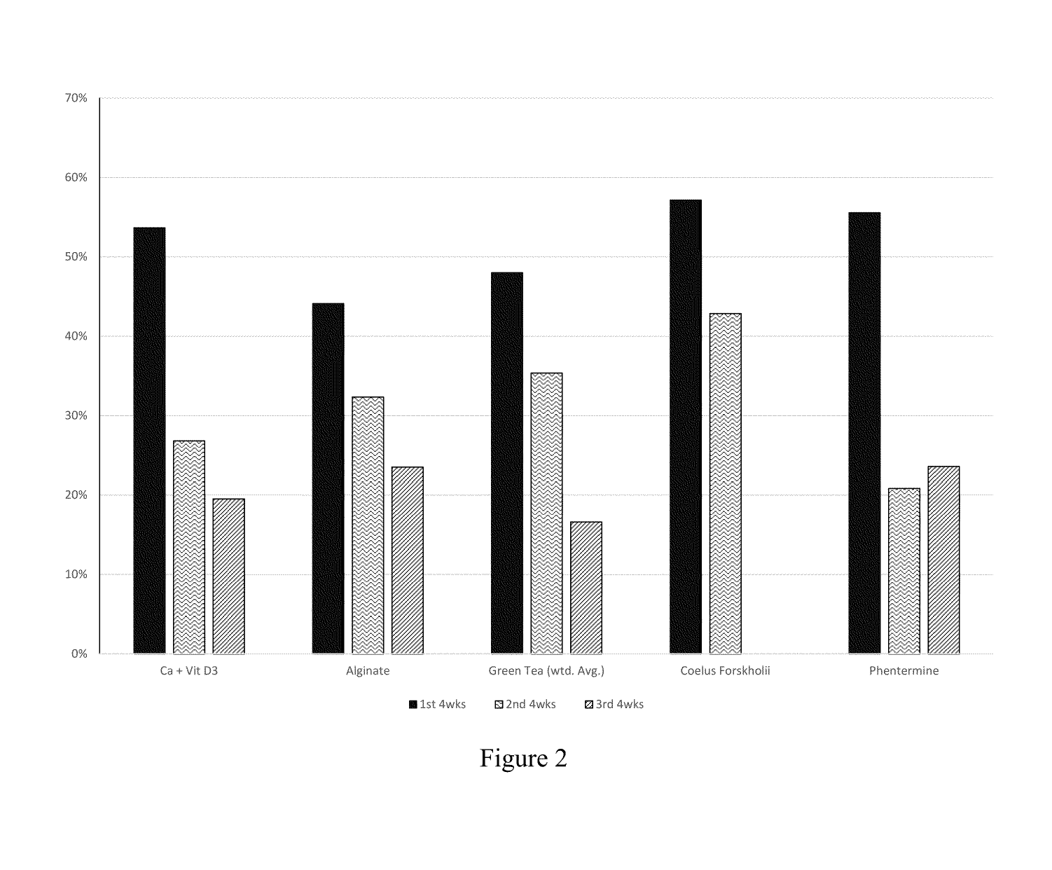

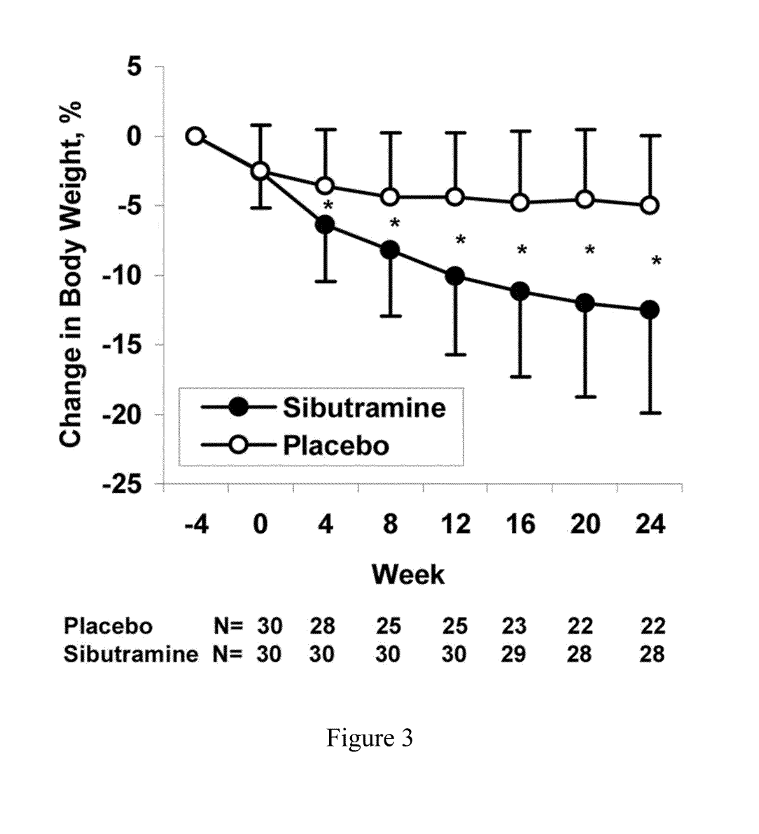

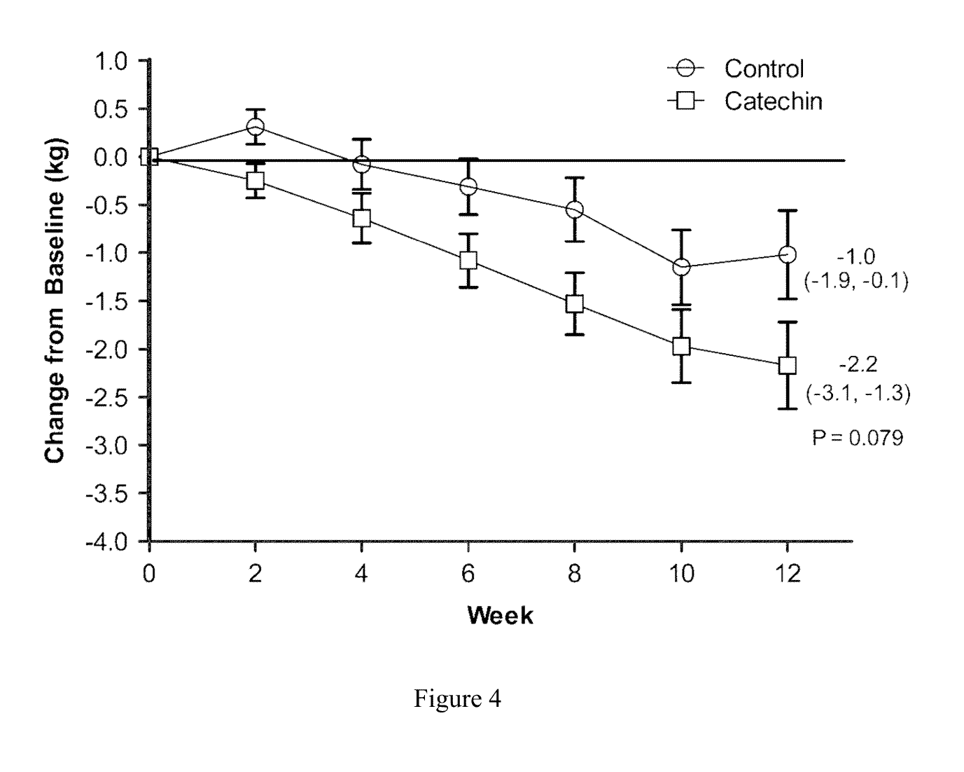

As can be seen in FIG. 1, subjects taking the weight loss agent Qysmia® (phentermine and topiramate extended-release capsules) lost weight during the first 28 weeks of the study, after which weight loss appears to have ceased. FIG. 1 also shows that the rate of weight loss decreases over time. When extended to a year, the average weight of the patients taking Qysmia® begins to increase after 26 weeks, suggesting that the agent is no longer effective in inducing weight loss in the subjects. The progressive decrease in the amount of weight lost as the treatment continues can also be seen in FIG. 2, where subsequent 4-week intervals produced less weight loss then the previous interval. FIGS. 3 and 4 show a steady weight loss during the first 24 or 12 weeks of the study, respectively, of particular weight loss agents. In another example, catecholamines may be used in weight loss due to their ability to induce lipolysis and increase glycolysis. Many of the physiological effects of catecholamines may be expressed via specific interaction with α-adrenergic and β-adrenergic receptors. β-adrenergic receptors are a part of a large family of GTP-binding regulatory protein (G-protein) mediated receptors. These membrane bound signaling protein complexes generally display the phenomenon of desensitization. Chronic administration of β-adrenergic agonists may lead to desensitization and/or a marked reduction in receptor density due to down regulation of the receptor genes. Similarly, phentermine has been shown to trigger the release of the neurotransmitter norepinephrine. Norepinephrine and its metabolite epinephrine are strong agonists of both α and β-adrenergic receptors. Through this activity, norepinephrine may cause appetite suppression, which in turn may result in weight loss. These receptors become desensitized to the elevated levels of norepinephrine, and their overall number is reduced. The end result may be a reduction or a complete cessation of weight loss after about 4 weeks. As a result, some weight loss agents may have a limited period of effectiveness, after which a subject may experience a weight loss plateau.

Some weight loss agents may modulate certain biological pathways. However, over time, the ability of a weight loss agent to modulate a particular pathway may be diminished or altered by changes in other biological pathways, thereby resulting in a decrease in the effect of the weight loss agent. In some cases, the weight loss agent may eventually lose its ability to effect weight loss due the changes in other biological pathways. For example, a certain weight loss agent may effect weight loss in a subject by inhibiting a particular biological pathway. Over time, the inhibition of this biological pathway may result in activation of other biological pathways that neutralize the effect of the weight loss agent and compensate for the inhibited pathway. In other circumstances, a subject can become desensitized to a particular weight loss agent over time such that the weight loss agent no longer modulates the biological pathway. By way of example, Phentermine, a prescription anorectic for the short-term treatment of obesity, has been shown to trigger the release of the neurotransmitter norepinephrine and cause appetite suppression, which in turn results in weight loss. However, over time, the body becomes desensitized to the elevated levels of norepinephrine in the central nervous system and appetite suppression is reduced. This effect may result in a reduction or even a complete cessation of weight loss after about 4 weeks, as can be seen in FIG. 2. As a result, many weight loss agents have a limited period of effectiveness after which the subject may experience a weight loss plateau.

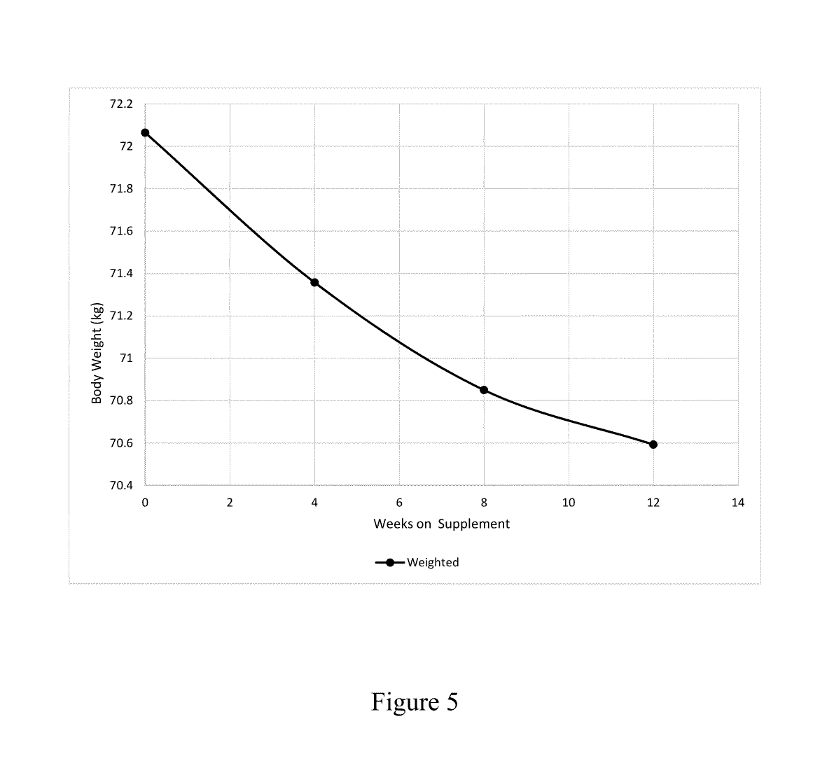

Camellia sinensis is a highly studied weight loss agent, thus suggesting that the point estimate of its effect is sound. The dose range of Camilla sinensis, is about 500 to about 700 mg per day. Pooling the data where the weight loss results were given in 4-week intervals, green tea supplementation appears to lose its effectiveness after 8 weeks as can be seen in FIG. 5.

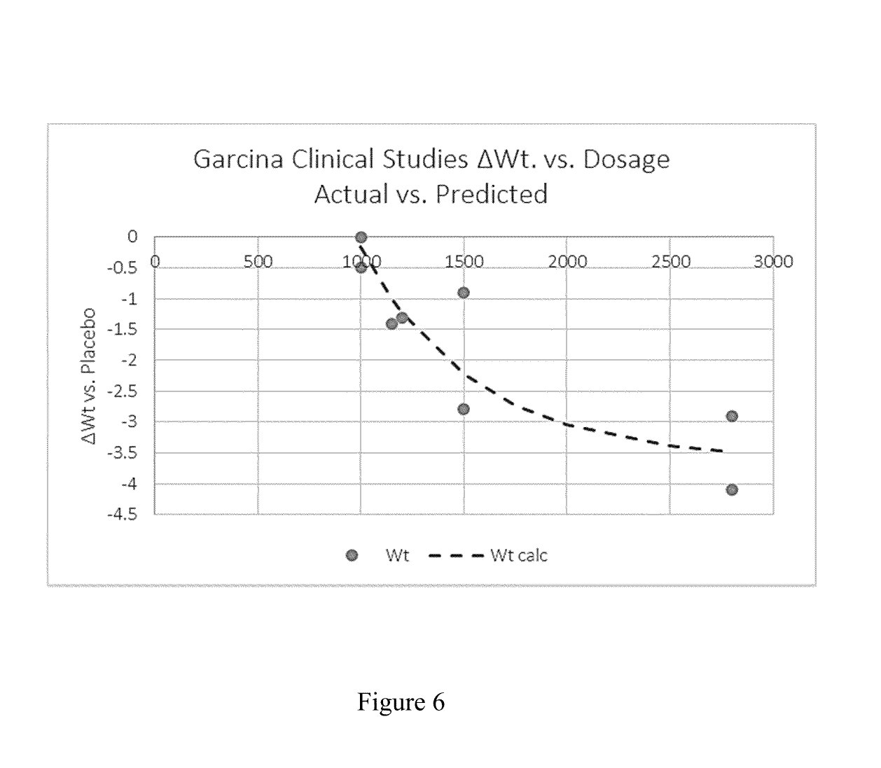

A systematic review and meta-analysis to examine the efficacy of Garcinia extract, hydroxycitric acid (HCA) as a weight reduction agent, using data from randomized clinical trials, found a small, statistically significant difference in weight loss of −0.9 kg between HCA and placebo. A nonlinear, significant (P<0.05) correlation between the dosage of HCA and body weight loss seems to exist. This relationship follows a curve ΔWt=ΔWtₘₐₓ(1−e^{-b*dosage}) which is consistent with a first order ligand binding kinetics. This data may suggest that HCA might produce are more pronounced effect at doses higher than 1500 mg/day. However, HCA quickly approaches its asymptotic maximum by 2800 mg/day. This data suggests that the ΔWtₘₐₓ for HCA is about 3.5 kg, and a dose of about 2000 mg/day would yield a more significant response of −3.0 kg over an 8-12 wk period, as shown in FIG. 6. Even at the higher doses, the weight loss effect is modest over this time period, and Garcina would likely need to be paired with another synergist agent to achieve weight loss more in line with 0.5 kg/week.

Disclosed herein are novel methods of effecting weight loss by improving a rate of weight loss and lengthening the time to the weight loss plateau. Such methods may include altering the mechanism of action of the different weight loss agent(s) used on a predetermined 1-20 week basis. By alternating the mechanisms of action, it may be possible to overcome the effects of the compensating mechanisms by actively treating these compensating mechanisms or attacking different therapeutic classes, which are currently not being affected.

Any of the compositions or kits described herein may be used to prolong weight loss. As described herein, some weight loss agents may lose effectiveness anywhere from about 4 to about 10 weeks after initiation of a weight loss program. This occurs once the body has compensated to the weight loss agent effect.

The term “weight loss,” as used herein, refers to any decrease in the weight, BMI or other measurement of mass of a subject. The term “subject,” as used herein, includes, but is not limited to, a person who is obese, a person who is overweight, a person who needs to or desires to decrease his/her weight slightly or a person who believes that modifying his/her weight would be beneficial. A “subject” may also mean a healthy individual who desires to lose weight. A “subject” may also mean an individual who has been diagnosed as having a condition that requires weight loss treatment.

As used herein, the terms “weight loss agent,” “active agent,” “therapeutic agent,” “therapeutic,” or “dietary supplement,” mean, respectively, a compound or composition used of prolong weight loss. Embodiments of the present disclosure may be directed to effecting weight loss, prolonging weight loss or a combination thereof. For example, in some embodiments, weight loss compositions and kits may have at least one weight loss agent. In some embodiments, weight loss compositions and kids may have a pharmaceutically or nutraceutically acceptable carrier or diluent, or an effective amount of a composition having at least one weight loss agent.

“Administering,” when used in conjunction with a weight loss agent, means to deliver a weight loss agent directly into or onto a target tissue or to deliver a therapeutic to a patient, whereby the therapeutic positively impacts the tissue to which it is targeted. Thus, as used herein, the term “administering,” when used in conjunction with a weight loss agent, can include, but is not limited to, providing a weight loss agent to a subject systemically by, for example, intravenous injection, whereby the therapeutic reaches the target tissue. Administering a composition may be accomplished by, for example, injection, oral administration, topical administration, or by these methods in combination with other known techniques. Such combination techniques may include heating, radiation, ultrasound and the use of delivery agents.

The term “therapeutic class” refers to the category or group that each weight loss agent is placed in based on known mechanisms of action. In some embodiments, the therapeutic class is selected from neurotransmitter modulators, hormonal satiety or hunger signaling modulators, β-adrenergic receptor modulators, α2-adrenergic modulators, adenosine receptor modulators, cannabinoid receptor modulators, protein-tyrosine phosphatase 1B modulators, catechol O-methyltransferase modulators, AMPk modulators, phosphodiesterase modulators, non-guanosine nucleotide mediated adenylate cyclase modulators, which may also be known as non-G-protein adenylate cyclase modulators, glucose metabolism modulators, carbohydrate metabolism modulators, lipid metabolism modulators, protein kinase A modulators, macro nutrient absorption modulators and agents with an unknown mechanism of action. In some embodiments, a weight loss agent can belong to more than one therapeutic class.

Particular weight loss agents may be classified within more than one therapeutic class. In some embodiments, the same therapeutic class may be administered sequentially, however the same weight loss agent may not be administered in sequential treatment phases.

The term “improves” is used to convey that the present invention changes either the characteristics and/or the physical attributes of the tissue to which it is being provided, applied or administered. The term “improves” may also be used in conjunction with a diseased state such that when a diseased state is “improved,” the symptoms or physical characteristics associated with the diseased state are diminished, reduced or eliminated. In some embodiments, “improved” is used to convey that the present invention changes either the characteristics and/or the attributes of the weight loss agent being used.

The term “inhibiting” includes the administration of a compound of the present invention to prevent the onset of the symptoms, alleviating the symptoms, or eliminating the disease, condition or disorder.

By “pharmaceutically acceptable” or “nutraceutically acceptable,” it is meant the carrier, diluent or excipient must be compatible with the other weight loss agents of the formulation and not deleterious to the recipient thereof or considered to generally recognized as safe (GRAS).

As used herein, the term “therapeutic” means an agent utilized to treat, combat, ameliorate, prevent or improve an unwanted condition or disease of a patient. In part, embodiments of the present invention are directed to the effecting weight loss in a subject, prolonging weight loss, or a combination thereof.

A “therapeutically effective amount” or “effective amount” of a composition is a predetermined amount calculated to achieve the desired effect, i.e., to effect weight loss, prolong weight loss, or a combination thereof. The activity contemplated by the present methods includes both medical therapeutic and/or prophylactic treatment, as appropriate. The specific dose of a weight loss composition comprising at least one weight loss agent administered according to this invention to obtain therapeutic and/or prophylactic effects will, of course, be determined by the particular circumstances surrounding the case, including, for example, the compound administered, the route of administration, and the condition being treated. However, it will be understood that the effective amount administered may be determined by physician or a person skilled in the art in the light of the relevant circumstances including the condition to be treated, the choice of compound to be administered, and the chosen route of administration, and therefore, the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

“Nutraceutical” as used herein generally refer to natural, bioactive chemical compounds that provide physiological benefits, including, disease prevention and health promotion which may be used to supplement the diet. Nutraceuticals can be either purified or concentrated by using bioengineering methods and can be enhanced through genetic methods, which contain elevated levels of natural substances. Examples of nutraceuticals include isolated nutrients and herbal products and generally contain at least one of the following ingredients: a vitamin, a mineral, an herb or other botanical, an amino acid, a metabolite, constituent, extract, or combination of these ingredients. Common examples of nutraceuticals include beta-carotene, ephedra, ginko biloba, goldenseal, valerian, ginseng, green tea extract, and echinacea. The nutraceuticals described herein may be useful for maintenance and support of, for example, healthy joints, skin, eye and brain function, heart and circulatory system, and general health.

As used herein, the term “consists of” or “consisting of” means that the formulation includes only the elements, steps, or weight loss agents specifically recited in the particular claimed embodiment or claim.

As used herein, the term “consisting essentially of” or “consists essentially of” means that the only active weight loss agent(s) in the formulation or method that treats the specified condition (e.g. nutrient depletion) is the specifically recited therapeutic(s) in the particular embodiment or claim.

As used herein the terms “modulation,” “modulate” or “modulating” as used refers to an increase, decrease, up-regulation, down-regulation, inhibition activation, or a combination thereof of a biological pathway.

Methods of Effecting Weight Loss

Embodiments herein are directed to a method of effecting weight loss in a subject. The method may comprise defining at least two treatment phases and administering at least one weight loss agent during each treatment phase. Each treatment phase may have a pre-determined duration wherein the at least one weight loss agent administered during each treatment phase may be selected from a different therapeutic class than the at least one weight loss agent administered during the preceding or proceeding treatment phase.

In some embodiments, the target weight loss goal may be about one pound or about 0.45 kg per week. In some embodiments, the loss of about one pound may be equivalent to a reduction in intake of about 3,500 calories. Accordingly, the magnitude of the effect may be roughly equivalent to a reduction in calorie intake by 500 calories per day from the subject's diet.

In some embodiments, the daily dose of the weight loss agent may be an effective amount. In some embodiments, the daily dose may be the per day Estimated Average Requirement amount. In some embodiments, the weight loss compositions described herein comprises each weight loss agent in an effective amount. In some embodiments, the weight loss compositions comprise each weight loss agent in an Estimated Average Requirement (EAR) amount. The Estimated Average Requirements may be the amount expected to satisfy the dietary needs of 50% of the people in a particular age group based on a review of the scientific literature. In some embodiments, the weight loss compositions comprise each weight loss agent in an Recommended Dietary Allowance amount. A Recommended Dietary Allowance (RDA) is the daily dietary intake level of a nutrient considered sufficient by the Food and Nutrition Board to meet the requirements of nearly all (97-98%) healthy individuals in each life-stage and gender group. In some embodiments, a weight loss compositions comprise each weight loss agent in an Adequate Intake amount. Adequate Intake (AI), where no RDA has been established may be adequate for each individual in a particular demographic group. In some embodiments, the weight loss compositions comprise each weight loss agent in an tolerable upper intake amount. Tolerable upper intake levels (UL), is the highest level of consumption that current data have shown to be safe for humans.

In some embodiments, the therapeutic class may be selected from a neurotransmitter modulator, a hormonal satiety or hunger signaling modulator, a β-adrenergic receptor modulators, a α2-adrenergic modulator, an adenosine receptor modulator, acannabinoid receptor modulator, a protein-tyrosine phosphatase 1B modulator, a catechol O-methyltransferase modulator, an AMPk modulator, a phosphodiesterase modulator, a non-guanosine nucleotide mediated adenylate cyclase modulator also known as a non-G-protein adenylate cyclase modulator, a glucose metabolism modulator, a carbohydrate metabolism modulator, a lipid metabolism modulator, a protein kinase A modulator, a macro nutrient absorption modulator and an agent with an unknown mechanism of action.

In some embodiments, neurotransmitter modulators may include, but are not limited to, agents that effect a change in synaptic transmission or concentration of a given neurotransmitter by modulating the release, production, function, re-uptake or a combination thereof, or neurotransmitters that are released by a neuron and in turn bind to and activate the receptors on another neuron or target cell. Without wishing to be bound by theory, neurotransmitter modulation can result in reduced appetite, an increase in metabolic activity, or a combination thereof, which may in turn lead to weight loss in a subject. In some embodiments, neurotransmitter modulators may include, but are not limited to, Catha edulis, Rhodiola rosea, Griffonia simplicifolia, Crocus sativus, Mucuna pruriens, L-theanine, Garcinia spp., Quercitin, and St. John's Wort.

In some embodiments, hormonal satiety or hunger signaling modulators may include, but are not limited to, agents that effect the production, release, degradation, function, or a combination thereof of a hormone involved in regulating food intake. Illustrative hormonal satiety or hunger signaling modulators may include, but are not limited to ghrelin, cholecystokinin, glucagon-like peptide-1, Peptide YY, and Neuropeptide Y, and the like. Without wishing to be bound by theory, such modulators may cause a change in feelings of satiety, feelings of hunger or a combination thereof, which may lead to decreased caloric intake, which in turn may lead to weight loss. In some embodiments, such weight loss agents may include, but are not limited to, macro nutrients such as soluble fiber, fatty acids, protein, Slendesta™, Fenugreek (Trigonella foenum-graecum), Plantago ovata (Plantago psyllium), Fibersol® 2, alginate, conjugated linoleic acid, α-lipoic acid, diacylglycerol, linolenate, Cocos nucifera oleum, whey protein and soy protein. In yet other embodiments, such weight loss agents may include, but are not limited to, herbal extracts of Coix lacryma-jobi, Ilex paraguariensis, Quecertin, Agave tequilana, Dasylirion sp., and Panax ginseng.

In some embodiments, β-adrenergic receptor modulators, α₂-adrenergic modulators, adenosine receptor modulators, and cannabinoid receptor modulators may include, but are not limited to, agents that modulate a biological response of a β-adrenergic receptor, an α₂-adrenergic receptor, an adenosine receptor, a cannabinoid receptor or a combination thereof. In some embodiments, a β-adrenergic receptor modulator, an α₂-adrenergic modulator, an adenosine receptor modulator, a cannabinoid receptor modulator may be an antagonist, an agonist or a combination thereof, of a β-adrenergic receptor, an α₂-adrenergic receptor, an adenosine receptor, a cannabinoid receptor or a combination thereof. In some embodiments, an antagonist, an agonist or a combination thereof, of a β-adrenergic receptor, an α₂-adrenergic receptor, an adenosine receptor, a cannabinoid receptor or a combination thereof may modulate the biological response of a β-adrenergic receptor, α₂-adrenergic receptor, adenosine receptor, cannabinoid receptor or a combination thereof by binding to a receptor and blocking normal biological response, binding to a receptor and triggering a biological response, or a combination thereof. Without wishing to be bound by theory, modulation of β-adrenergic receptors, α₂-adrenergic receptors, adenosine receptors, cannabinoid receptors or a combination thereof may result in increased metabolic activity, which in turn may lead to weight loss. Without wishing to be bound by theory, activation of β-adrenergic receptors may result in increased metabolic activity through activation of adenylate cyclase, which in turn may lead to weight loss. Without wishing to be bound by theory, inhibition of α₂-adrenergic receptors may result in increased metabolic activity through an increase in adenylate cyclase activity, which in turn may lead to weight loss. Without wishing to be bound by theory, two sub-types of adenosine receptors, namely A₁ and A₃, may modulate adenylate cyclase. Blocking these adenosine receptors may result in increased metabolic activity, which in turn may lead to weight loss. In some embodiments, β-adrenergic receptor agonists, α2-adrenergic antagonist, and adenosine receptor agonists/antagonists may effect adenylate cyclase via G-protein signaling. Without wishing to be bound by theory, the a subunit of the G-protein complex may release upon activation of the receptor. The Gas subunit connected with the β-adrenergic and the adenosine A₁ and A₃ receptors may activate adenylate cyclase, while the Gαi subunit attached to the α2-adrenergic and adenosine A2 receptor inhibit adenylate cyclase. Adenylate cyclase converts ATP into cAMP, which then activates Protein Kinase A (PKA). PKA increases the metabolic rate by promoting glycolysis and lipolysis, which in turn may lead to weight loss. Studies have shown that Citrus aurantium and Capsicum sp. modulate β-adrenergic receptors. Yohimbine and Rubus idaeus have also been shown to inhibit the α2 receptor. Caffeine and xanthine alkaloids have been shown to inhibit adenosine receptors. In some embodiments, cannabinoid receptor modulators may include, but are not limited to, PhosphoLean®. In some embodiments, β-adrenergic receptor modulators may include, but are not limited to, Citrus aurantium and Capsicum sp., also known as capsinoids. In some embodiments, α₂ receptor modulators may include, but are not limited to, yohimbine and Rubus idaeus. In some embodiments, adenosine receptor modulators may include, but are not limited to, caffeine, xanthine alkaloids, Guarana sp., Ilex paraguariensis, and theobromine. In some embodiments, certain neurotransmitter modulators and catechol O-methyltransferase modulators may also modulate the β-adrenergic receptors including, but not limited to, Catha edulis, Rhodiola rosea, Crocus sativus, St. John's Wort., Camellia sinensis and PhosphoLean®.

In some embodiments protein-tyrosine phosphatase 1B (PTP1B) modulators may refer to, but are not limited to, agents that modulate PTP1B, a negative regulator of the insulin and leptin signaling. Without being bound by theory, insulin promotes the uptake of glucose into tissue, where it is stored as glycogen and triglycerides, which in turn can result in weight gain. Inhibition of the effects of insulin on glucose uptake may result in weight loss. In addition, the peptide hormone leptin plays a central role in feeding and adiposity. As plasma leptin levels fall, appetite is stimulated and energy expenditure is reduced. When leptin levels increase, appetite is suppressed and energy expenditure is increased. Human obesity is associated with high circulating levels of leptin, suggesting that resistance to the effects of leptin may underlie most types of obesity. In some embodiments, modulation of PTP1B may result in increased leptin sensitivity and weight loss. In some embodiments, modulators of PTP1B include, but are not limited to, Cinnamomum cassia.

In some embodiments, catechol O-methyltransferase (COMT) modulators may refer to, but are not limited to, agents that effect the function of COMT, which is an enzyme, involved in the degradation of catecholamines such as dopamine, epinephrine, and norepinephrine into inactive forms. In some embodiments, catechol O-methyltransferase (COMT) modulators may be COMT antagonists. Without wishing to be bound by theory, catechol O-methyltransferase antagonists may modulate peripheral neuronal responses, which may result in increases in metabolism and weight loss. Without wishing to be bound by theory, inhibitors of COMT may increase the amount of circulating catecholamines, which are strong agonists of β-adrenergic receptors. COMT inhibitors may also increase metabolism via β-adrenergic pathway. In some embodiments, inhibitors of COMT include, but are not limited to, Camellia sinensis.

In some embodiments, 5′ AMP-activated protein kinase (AMPk) modulators may refer to, but are not limited to, agents that induce the activation of AMPk. Without wishing to be bound by theory, AMPK is a key regulator in metabolism and cellular energy homeostasis. This protein is highly conserved with little variation from yeast to humans. AMPK is expressed in numerous tissues including brain, liver, and skeletal muscle. AMPK activation leads to hepatic fatty acid oxidation, inhibition of cholesterol synthesis, inhibition of lipogenesis, inhibition of triglyceride synthesis, inhibition of adipocyte lipolysis and lipogenesis, stimulation of skeletal muscle fatty acid oxidation, and muscle glucose uptake. Activation of AMPK may also be involved in cellular energy homeostasis and regulating lipid metabolism, which may result in weight loss. In some embodiments, modulators of AMPk include Salix alba, Coffea canefora, Ilex paraguariensis, Ecklonia cava, phillyrin, Rubus idaeus, and Panax ginseng berry and root.

In some embodiments, phosphodiesterase-modulating agents may include, but are not limited to, agents that inhibit the degradation of cyclic adenosine monophosphate (cAMP), which is used for intracellular signal transduction. Without wishing to be bound by theory, phosphodiesterase is an enzyme that catalyzes the reaction, which converts cyclic adenosine phosphate (cAMP) to adenosine monophosphate (AMP). Phosphodiesterase (PDE) inhibitor blocks the degradation of cyclic adenosine monophosphate (cAMP), which is used for intracellular signal transduction. In some embodiments, phosphodiesterase modulators may increase levels of cAMP by limiting its degradation. cAMP activates PKA. PKA increases the metabolic rate by promoting glycolysis and lipolysis. Without wishing to be bound by theory, increasing the cAMP levels may increase metabolism and may lead to weight loss. In some embodiments, phosphodiesterase modulating agents include Sinetrol®, phillyrin, caffeine, Guarana, Ilex paraguariensis and theobromine.

In some embodiments, non-guanosine nucleotide mediated adenylate cyclase, also known as non-G-protein adenylate cyclase modulators, modulating agents may refer to, but are not limited to, agents that effect the function of adenylate cyclase, which is an enzyme involved in the production of cAMP in a manner that is independent from the guanosine nucleotide-signaling pathway. It is believed that the vast majority of production of cAMP is created through the guanosine nucleotide protein complex (G-protein) mediated activation of adenylate cyclase. Without wishing to be bound by theory, increasing the cAMP levels will increase metabolism, which may lead to weight loss. In some embodiments, adenylate cyclase modulators include, but are not limited, to Coleus forskholii.

Glucose metabolism modulators may effect the absorption, digestion or a combination thereof, of carbohydrates. These agents predominately act as inhibitors of α-amylase, α-glucosidase, or both. Both α-amylase, α-glucosidase are involved in the breakdown of polysaccharides (starches) into monosaccharides (simple sugars) in the human digestive system. Since the human body is only able to absorb monosaccharides, inhibiting these enzymes may decrease the amount of calories absorbed for a given ingestion of carbohydrates, and by doing so, this may lead to weight loss. In some embodiments, glucose metabolism modulators may refer to, but are not limited to, agents that cause an increase glycolysis, which may result in weight loss. In some embodiments, carbohydrate metabolism modulators may refer to, but are not limited to, agents that effect the absorption and/or breakdown of carbohydrates. Without wishing to be bound by theory, a decrease in the absorption or digestion of carbohydrates may lead to weight loss. In some embodiments, glucose metabolism modulators include but are not limited to Momordica charantia, Olea europea and Theobroma cacao bean. In some embodiments, carbohydrate metabolism modulators include, but are not limited to, Garcinia sp., Coffea canefora, Phaseolus vulgaris, Cissus quadrangularis, Irvingia gabonensis, Carthamus tinctorius, Laminaria japonica, Curcuma longa, Zingiber officinale, Rosemarinus officinalis and Gymnema sylvestre.

In some embodiments, lipid metabolism modulators may refer to, but are not limited to, agents that effect the absorption, production, and/or breakdown, of lipids. These agents predominately act by inhibiting pancreatic lipase. Pancreatic lipase is secreted from the pancreas, and hydrolyzes dietary fat molecules in the human digestive system. Pancreatic lipase converts triglyceride substrates (fats) ingested oils into mono-glycerides and free fatty acids, which the human body is able to absorb. Alli® (orlistat) is an example of this class of compounds. Alli® demonstrated that the decreased absorption and digestion of lipids led to weight loss. Without wishing to be bound by theory, decreased absorption, digestion, or production of lipids may lead to weight loss. In some embodiments, lipid metabolism modulators may include, but are not limited to, Isohumulones, also known as Humulus lupulus, Arachis hypogaea, Allium victorialis, Punica granatum leaf, Kochis scoparia, Panax japonicas, Aesculus turbinate, Ceratonia siliqua pulp, oolong tea, Ilex paraguariensis, Glycyrrhiza glabra and Cissus quadrangularis.

In some embodiments, protein kinase A (PKA) modulators may refer to, but are not limited to, agents that effect enzymes whose activity is dependent on cellular levels of cyclic AMP (cAMP). Activated PKA can then catalyze the transfer of ATP terminal phosphates to protein substrates at serine, or threonine residues. This phosphorylation may result in a change in activity of the substrate. Since PKAs are present in a variety of cells and act on different substrates, PKA regulation and cAMP regulation may be involved in many different pathways.

In some embodiments, agents with an unknown mechanism of action may refer to, but are not limited to, agents that have yet to be places in a known therapeutic class but have been shown to have an effect on weight loss. In some embodiments, agents with an unknown mechanism may include, but are not limited to, Caralluma fimbriata, Hoodia gordonii, Parasitic loranthus, Paeonia suffruticosa, Turnera diffusa, Sambuca nigra, Asparagus officinalis, 5-hydroxytryptophan, Slendesta®, Olea europea and acetic acid (vinegar).

In some embodiments, neurotransmitter modulators may be selected from, but are not limited to, Griffonia simplicifolia, Crocus sativus, Mucuna pruriens, L-theanine, St. John's Wort, Catha edulis, Rhodiola rosea, Quercitin, and Garcina sp. In some embodiments, hormonal satiety or hunger signaling modulators may be selected from, but are not limited to, Slendesta™, Fenugreek (Trigonella foenum-graecum), Plantago ovata (Plantago psyllium), Fibersol® 2, alginate, conjugated linoleic acid, α-lipoic acid, diacylglycerol, linolenate, Cocos nucifera oleum, whey protein, soy protein, Coix lacryma-jobi, Ilex paraguariensis, Quercitin, Agave tequilana, Dasylirion sp., and Panax ginseng. In some embodiments, β-adrenergic receptor modulators may be selected from, but are not limited to, Citrus aurantium and Capsicum sp., also known as capsinoids. In some embodiments, α₂-adrenergic receptor modulators may be selected from, but are not limited to, yohimbine and Rubus idaeus. In some embodiments, adenosine receptor modulators may be selected from, but are not limited to, caffeine, Guarana sp., Ilex paraguariensis, xanthine alkaloids, and theobromine. In some embodiments, cannabinoid receptor modulators may be selected from, but are not limited to, PhosphoLean®. In some embodiments, protein-tyrosine phosphatase 1B (PTP1B) modulators may be selected from, but are not limited to, Cinnamomum cassia. In some embodiments, catechol O-methyltransferase (COMT) modulators may be selected from but are not limited to, Camellia sinensis and PhosphoLean®. In some embodiments, 5′ AMP-activated protein kinase (AMPk) modulators may be selected from, but are not limited to, Salix alba, Coffea canefora, Ilex paraguariensis, Ecklonia cava, phillyrin, Rubus idaeus, and Panax ginseng berry and root. In some embodiments, phosphodiesterase modulators may be selected from, but are not limited to, Sinetrol®, phillyrin, caffeine, Guarana sp., Ilex paraguariensis and theobromine. In some embodiments, non-guanosine nucleotide mediated adenylate cyclase modulators, also known as non-G-protein adenylate cyclase modulators, may be selected from, but are not limited to, Coleus forskholii. In some embodiments, glucose metabolism modulators may be selected from, but are not limited to, Momordica charantia, Olea europea and Theobroma cacao bean. In some embodiments, carbohydrate metabolism modulators may be selected from, but are not limited to, Garcinia sp., Coffea canefora, Phaseolus vulgaris, Cissus quadrangularis, Irvingia gabonensis, Carthamus tinctorius, Laminaria japonica, Curcuma longa, Zingiber officinale, Rosemarinus officinalis and Gymnema sylvestre. In some embodiments, lipid metabolism modulators may be selected from, but are not limited to, Isohumulones, also known as Humulus lupulus, Arachis hypogaea, Allium victorialis, Punica granatum leaf, Kochis scoparia, Panax japonicas, Aesculus turbinate, Ceratonia siliqua pulp, oolong tea, Ilex paraguariensis, Glycyrrhiza glabra, and Cissus quadrangularis. In some embodiments, agents with an unknown mechanism of action may be selected from, but are not limited to, Caralluma fimbriata, Hoodia gordonii, Parasitic loranthus, Paeonia suffruticosa, Turnera diffusa, Sambuca nigra, Asparagus officinalis, 5-hydroxytryptophan, Slendesta®, Olea europea and acetic acid (vinegar).

In some embodiments, the weight loss agents described here may be configured as individual doses. In some embodiments, the weight loss agents may be configured for daily administration. In some embodiments, the individual dose of the weight loss agent is configured to include a single daily dose. In some embodiments, the individual doses of a weight loss agent comprise a fraction of the daily dose such as, but not limited to, half the daily dose for administration twice a day or a third of the daily dose for administration three times a day. In yet other embodiments, the individual doses can be configured in any suitable combination to provide the desired daily dose of a weight loss agent.

The administration of green tea (Camellia sinensis) catechins (GTCs) with caffeine is associated with statistically significant reductions in body mass index (BMI), body weight, and Waist Circumference (WC). It has been found that catechin-caffeine mixtures or a caffeine-only supplementation stimulates daily energy expenditure dose-dependently by about 0.10-0.13 Kcal/mg administered. Compared with placebo, daily fat-oxidation was only significantly increased by catechin-caffeine mixtures. COMT plays as significant role in a wide variability in flavonoid metabolism. The inter-individual variability of the activity of COMT could vary as much as three-fold. COMT enzyme activity has been reported to differ between ethnic groups. A dosage of 600 mg of epigallocatechin gallate (ECGC) contains a total of 960 mg of catechins. In some embodiments, 960 mg of catechins may elevate the resting metabolic rate by about 122 Kcal/day. In some embodiments, the daily dose of Camellia sinensis may be about 400 mg to about 1,400 mg. In some embodiments, the daily dose of Camellia sinensis may be about 500 to about 700 mg. In some embodiments, the daily dose of Camellia sinensis may be about 600 mg. In some embodiments, Camellia sinensis may be administered three times a day at a dose of 200 mg ECGC.

Capsaicinoids are a group of chemicals found in all members of the Capsicum genus (chili peppers). Capsaicinoids have been linked to increased β-adrenergic stimulation. Consumption of capsaicinoids increases energy expenditure by about 50 kcal/day to about 100 kcal/day and regular consumption significantly reduced abdominal adipose tissue levels and reduced appetite and energy intake. In some embodiments, the specific Casicum sp. to be administered may be Capsicum annum. In some embodiments, the daily dose of Capsicum sp. may be 7.5 mg. In some embodiments, Capsicum sp. may be administered three time per day at a dose of 2.5 mg. In some embodiments, the daily dose of Capsicum sp. may be about 30 mg to about 150 mg. In some embodiments, the daily dose of Capsicum sp. may be about 10 mg to about 30 mg. In some embodiments, the daily dose of Capsicum sp. may be about 15 mg to about 30 mg. In some embodiments, the daily dose of Capsicum sp. may be about 7.5 mg to about 150 mg. In some embodiments, the daily dose of Capsicum sp. may be about 450 mg of 40,000 SHU (0.25% capsaicin).

Garcinia extract, which is hydroxycitric acid (HCA) used as a weight reduction agent, demonstrates a statistically significant difference in weight loss of about −0.9 kg between HCA and placebo. HCA may produce a more pronounced effect at doses higher than about 1500 mg/day. However, HCA quickly approaches its asymptotic maximum by about 2800 mg/day. The ΔWtmax for HCA is about 3.5 kg, and a dose of about 2000 mg/day would yield a more significant response of about −3.0 kg over an 8-12 week period. Even at the higher doses, the weight loss effect is modest over this time period. In some embodiments, Garcina may be paired with another synergist agent to achieve weight loss of about 0.5 kg/week. In some embodiments, the daily dose of Garcinia sp. may be about 500 mg to about 3,000 mg. In some embodiments, the daily dose of Garcinia sp. may be about 1500 mg. In some embodiments, the daily dose of Garcinia sp. may be about 2800 mg. In some embodiments, the daily dose of Garcinia sp. may be about 2000 mg.

Citrus aurantium may cause a slight rise in the resting metabolic rate (RMR) of about 40 kcal/day. This increase in RMR may yield about 1 kg of weight loss after 12 weeks of treatment, assuming the effect was constant over that entire period. In some embodiments, Citrus aurantium may be paired with other weight loss agents to achieve optimal weight loss. In some embodiments, the daily dose of Citrus aurantium may be determined by one skilled in the art to achieve about 10 mg to about 70 mg of p-Synerphine. In some embodiments, the daily dose of Citrus aurantium may be about 50 mg.

Phaseolus vulgaris is an α-amylase inhibitor. α-amylase is a key enzyme in carbohydrate digestion. Some studies have included a diet with normal to above normal amounts of carbohydrates. It stands to reason that people following an enriched carbohydrate diet would benefit more than those following a low carbohydrate diet. In some embodiments, Phaseolus vulgaris may be combined with another agent such as pancreatic lipase inhibitor to help mitigate some of the potential variability caused by diet. In some embodiments, the daily dose of Phaseolus vulgaris may be about 2,000 amu² to about 3,000 amu² (amu² stands for alpha-amylase blocking units). In some embodiments, the daily dose of Phaseolus vulgaris may be determined by one skilled in the art. In some embodiments, the daily dose of Phaseolus vulgaris may be about 300 mg. In some embodiments, the daily dose of Phaseolus vulgaris may be in the form of 1500 mg of Phase 2® Starch Neutralizer administered twice daily.

Caffeine increases resting metabolic rate by increasing energy expenditure. In some embodiments, a 600 mg/day dose of caffeine may raise energy expenditure by 63 Kcal/day. 100 mg appears to be the minimum (or near minimum) dose that would allow a demonstrable stimulatory effect on resting metabolic rate (RMR). In addition, the thermogenic effect of caffeine in man is dose dependent. In some embodiments, the magnitude of response increases linearly with higher doses, and the thermogenic responses to doses of 100, 200-250, and 400-450 mg were 4-5, 10-12, and 16%, respectively. In some embodiments, the daily dose of caffeine may be less than about 1,500 mg up to about 500 mg. In some embodiments, the daily dose of caffeine may be about 100 mg. In some embodiments, the daily dose of caffeine may be about 200 mg to about 250 mg. In some embodiments, the daily dose of caffeine may be about 400 mg to about 450 mg. In some embodiments, the daily dose of caffeine may be about 500 mg to about 700 mg. In some embodiments, the daily dose of caffeine may be about 600 mg. In some embodiments, the daily dose of caffeine may be about 400 mg. In some embodiments, the daily dose of caffeine may be about 100 mg to about 1500 mg.

Yohimbine has high affinity for the α2-adrenergic receptor and moderate affinity for the α1-receptor, where it functions as an antagonist. In some embodiments, the daily dose of Pausinystalia yohimbe may be about 333 mg to about 1,000 mg. In some embodiments, the daily dose of yohimbine may be about 20 mg. In some embodiments, the daily dose of yohimbine may be about 7.5 mg to about 10 mg. In some embodiments, the daily dose of yohimbine may be about 7.5 mg to about 1,000 mg.

Ilex paraguariensis may reduce heart rate and respiratory quotient (a measure of fat oxidation). Zotrim®, which is a fixed blend of Ilex paraguariensis, Paullinia cupana, Turnera diffusa, which may lead to weight loss and fewer consumed calories. In some embodiments, the daily dose of Ilex paraguariensis, in the form of Zotrim®, may be a single commercial dose. In some embodiments, the daily dose of Ilex paraguariensis may be about 100 to about 3,000 mg.

5-hydroxytryptophan (5-HTP) has an effect on weight loss that is facilitated by a spontaneous reduction on calorie consumption. In some embodiments, the effect of 5-HTP may be equivalent to a reduction in energy intake of energy intake of about 243 Kcal/day. In some embodiments, the daily dose of 5-HTP may be administered orally at doses of about 750 mg to about 900 mg. In some embodiments, the daily dose of 5-HTP may be administered by oral spray at about 50 mg. 5-HTP, which ingested, can cause nausea and diarrhea. Therefore, there are advantages to delivering the 5-HTP via the oral cavity. In some embodiments, 5-HTP may be administered as a quick dissolve tablet. In some embodiments, the daily dose of 5-HTP may be administered sublingually at about 300 mg. In some embodiments, the daily dose of 5-HTP may be about 50 mg to about 900 mg.

Potato protease inhibitor (PI2) and Slendesta® Potato Extract 5% Powder reduce body weight. In some embodiments, potato protein inhibitor (PI2) may be administered at about 15 to about 30 mg twice daily. In some embodiments, Slendesta® Potato Extract 5% Powder may be administered at about 300 mg to about 600 mg twice daily. In some embodiments, the daily dose of Slendesta® may be between about 15 mg and about 30 mg.

In some embodiments, Coleus forskholii may be administered alone. In some embodiments, Coleus forskholii may be administered in combination with other agents. Forskolin, the active component in Coleus forskholii, is widely recognized a G-protein independent activator of adenylate cyclase, and as such, it may be useful as an adjunct to other agents to increase their effectiveness. Forskolin has been shown to potentiate the effects of the beta-adrenergic agonist isoproterenol, and has shown dose dependent benefits in increasing cAMP alongside isoproterenol. In situations where beta-adrenergic agonists do not stimulate (hyporesponsiveness), low doses of forskolin were shown to rescue the effectiveness of beta-adrenergic agonists in the skin. 1 μM forskolin (although not lesser concentrations) are able to rescue beta-adrenergic desensitization in vitro. Forskolin is also synergistic with methylxanthines (caffeine, and/or the like), as methylxanthines have the ability to reduce adenosine's suppressive influence on elevated cAMP levels in adipocytes via acting as adenosine inhibitors. In some embodiments, the daily dose of Coleus forskholii may be about 25 mg to about 75 mg. In some embodiments, the daily dose of Coleus forskholii may be about 50 mg. In some embodiments, the daily dose of Coleus forskholii may be about 25 mg. In some embodiments, the daily dose of Coleus forskholii may be about 250 mg to about 750 mg of an extract of about 10% Coleus forskholii. In some embodiments, the daily dose of Coleus forskholii may be about 125 mg to about 375 mg of an extract of about 20% Coleus forskholii.

In some embodiments, the daily dose of Cissus quadrangularis may be about 200 mg to about 600 mg. In some embodiments, the daily dose of Cissus quadrangularis may be about 300 mg.

In some embodiments, Irvingia gabonensis may be administered alone. In some embodiments, the daily dose of Irvingia gabonensis may be about 150 mg. In some embodiments, the daily dose of Irvingia gabonensis may be about 150 to about 500 mg. In some embodiments, the daily dose of Irvingia gabonensis may be about 200 to about 500 mg. In some embodiments, the daily dose of Irvingia gabonensis may be about 300 mg. In some embodiments, the daily dose of Irvingia gabonensis may be in the form of 150 mg of Integra-Lean® African Mango administered twice daily.

PhosphoLean® contains a complex of oily N-oleyl-phosphatidylethanolamine (NOPE) and epigallocatechin-3-gallate (EGCG). NOPE-EGCG treatment improved the sensation of fullness and severity of binge eating. In some embodiments, the daily dose of PhosphoLean® may be a capsule containing 85 mg NOPE and 50 mg EGCG. In some embodiments, the daily dose of PhosphoLean® may be a capsule containing 120 mg NOPE and 105 mg of EGCG. In some embodiments, PhosphoLean® may be administered as a single daily dose.

In some embodiments, Ecklonia cava may be administered as Xanthigen, a combination of brown marine algae fucoxanthin and pomegranate seed oil (PSO)). In some embodiments, a daily dose of Fucoxanthin may be greater than 2.4 mg. In some embodiments, a daily dose of Xanthigen-400/1.6 mg may be about 200 mg PSO and about 200 mg brown seaweed extract containing 1.6 mg fucoxanthin. In some embodiments, the daily dose of Ecklonia cava may be about 150 mg. In some embodiments, the daily dose of Ecklonia cava may be about 144 mg. In some embodiments, the daily dose of Ecklonia cava may be administered at a dose greater than 1.6 mg.

In some embodiments, Sinetrol® may be administered in the form of Sinetrol-XPur (polyphenolic citrus dry extract). In some embodiments, the daily dose of Sinetrol® may be about 900 mg to about 1,400 mg. In some embodiments, the daily dose of Sinetrol® may be administered as a single daily dose.

Licorice may suppress production of aldosterone without any change in BMI. Additionally, licorice may reduce fat by inhibiting 11 β-hydroxysteroid dehydrogenase Type 1 at the level of fat cells. In some embodiments, Glycyrrhiza uralensis may be administered in the form of licorice flavonoid oil. In some embodiments, the daily dose of Glycyrrhiza uralensis, in the form of licorice flavonoid oil, may be about 300 mg. In some embodiments, Glycyrrhiza uralensis may be administered in the form of a commercial preparation of licorice. In some embodiments, the daily dose of Glycyrrhiza uralensis, in the form of licorice, may be about 3.5 g. In some embodiments, the daily dose of Glycyrrhiza uralensis may be determined by one skilled in the art.

In some embodiments, the daily dose of St. John's Wort may be about 300 mg. In some embodiments, the daily dose of St. John's Wort may be about 27 mg. In some embodiments, the daily dose of St. John's Wort may be about 27 mg to about 300 mg.

In some embodiments, Olea europea may be administered as olive leaf polyphenols. In some embodiments, the daily dose of Olea europea may be 51.1 mg oleuropein, and 9.7 mg hydroxytyrosol. In some embodiments, the daily dose of Olea europea may be determined by one skilled in the art. In some embodiments, the daily dose of Olea europea may be about 150 mg.

The mechanism of action of Rhodiola rosea is as a non-specific Mono-oxidase inhibitor (MOAI) that helps to maintain elevated levels of norepinephrine and serotonin in the brain leading to continued appetite suppression. In some embodiments, the daily dose of Rhodiola rosea may be about 50 mg to about 200 mg. In some embodiments, the daily dose of Rhodiola rosea may be about 300 mg to about 600 mg. In some embodiments, the daily dose of Rhodiola rosea may be about 500 mg. In some embodiments, the daily dose of Rhodiola rosea may be about 100 mg. In some embodiments, the daily dose of Rhodiola rosea may be about 576 mg. In some embodiments, the daily dose of Rhodiola rosea may be about 340 mg. In some embodiments, the daily dose of Rhodiola rosea may be about 680 mg. In some embodiments, the daily dose of Rhodiola rosea may be about 200 mg. In some embodiments, the daily dose of Rhodiola rosea may be about 50 mg to about 680 mg.

In some embodiments, the daily dose of Crocus sativus may be about 30 mg. In some embodiments, the daily dose of Crocus sativus may be about 176.5 mg. In some embodiments, the daily dose of Crocus sativus may be about 30 mg to about 176.5 mg.

In some embodiments, the daily dose of Coffea canefora may be between about 300 mg to about 500 mg. In some embodiments, the daily dose of Coffea canefora may be administered as the commercial preparation, Svetol®. In some embodiments, the daily dose of Coffea canefora may be about 400 mg. In some embodiments, the daily dose of Coffea canefora may be about 1600 mg, standardized to 50% total chlorogenic acids. In some embodiments, the daily dose of Coffea canefora may be between about 700 mg to about 1050 mg. In some embodiments, the daily dose of Coffea canefora may be about 200 mg. In some embodiments, the daily dose of Coffea canefora may be between about 200 mg to about 1600 mg.

In some embodiments, the daily dose of Panax ginseng may be about 5,000 mg to about 20,000 mg. In some embodiments, the daily dose of Panax ginseng berry may be about 1,500 mg (20 mg per kg) to about 10,500 mg (150 mg per kg). In some embodiments, Panax ginseng may be about 1,500 mg to about 20,000 mg.

In some embodiments, the daily dose of Catha edulis may be determined by one skilled in the art. In some embodiments, the daily dose of Guarana sp. (Paullinia cupana) may be determined by one skilled in the art to achieve less than about 1,500 mg of caffeine. In some embodiments, the daily dose of Griffonia simplicifolia may be about 150 to about 900 mg. In some embodiments, the daily dose of Mucuna Pruriens may be determined by one skilled in the art to achieve 60-120 mg of total L-dopa. In some embodiments, the daily dose of L-theanine may be about 200 mg to about 800 mg. In some embodiments, the daily dose of Slendesta™ may be about 30 mg to about 90 mg. In some embodiments, the daily dose of fenugreek (Trigonella foenum-graecum) may be greater than about 1,000 mg. In some embodiments, the daily dose of Plantago ovata (Plantago psyllium) may be greater than 1,000 mg. In some embodiments, the daily dose of Fibersol® 2, may be greater than 1,000 mg. In some embodiments, the daily dose of alginate may be greater than 1,000 mg. In some embodiments, the daily dose of conjugated linoleic Acid may be about 500 mg to about 4,000 mg. In some embodiments, the daily dose of α-lipoic acid may be about 200 mg to about 2,000 mg. In some embodiments, the daily dose of diacylglycerol may be about 1,000 mg to about 10,000 mg. In some embodiments, the daily dose of linolenate may be about 890 mg. In some embodiments, the daily dose of Cocos nucifera oleum may about 24,000 mg to about 27,000 mg. In some embodiments, the daily dose of whey protein may be about 1,000 mg to about 60,000 mg. In some embodiments, the daily dose of soy protein may about 1,000 mg to about 60,000 mg. In some embodiments, the daily dose of Coix lacryma-jobi may be determined by one skilled in the art. In some embodiments, the daily dose of Agave tequilana may be greater than 1,000 mg. In some embodiments, the daily dose of Quecertin may be greater than 1,000 mg. In some embodiments, the daily dose of Rubus idaeus may be determined by one skilled in the art. In some embodiments, the daily dose of Cinnamomum cassia may be about 1,000 mg to about 6,000 mg. In some embodiments, the daily dose of Salix alba may be determined by one skilled in the art to achieve 15-120 mg of total salicin. In some embodiments, the daily dose of phillyrin may be determined by one skilled in the art. In some embodiments, the daily dose of Theobromine may be about 1,000 mg. In some embodiments, the daily dose of Sphaerantus indicus may be about 800 mg. In some embodiments, the daily dose of Momordica charantia may be determined by one skilled in the art. In some embodiments, the daily dose of Theobroma cacao bean may be determined by one skilled in the art. In some embodiments, the daily dose of Carthamus tinctorius may be determined by one skilled in the art. In some embodiments, the daily dose of Laminaria japonica may be determined by one skilled in the art. In some embodiments, the daily dose of Curcuma longa may be determined by one skilled in the art. In some embodiments, the daily dose of Zingiber officinalis may be determined by one skilled in the art. In some embodiments, the daily dose of Gymnema sylvestre may be about 400 mg. In some embodiments, the daily dose of Isohumulones may be about 20 mg to about 60 mg. In some embodiments, the daily dose of Arachis hypogaea may be determined by one skilled in the art. In some embodiments, the daily dose of Allium victorialis may be determined by one skilled in the art. In some embodiments, the daily dose of Punica granatum leaf may be determined by one skilled in the art. In some embodiments, the daily dose of Kochis scoparia may be determined by one skilled in the art. In some embodiments, the daily dose of Panax japonicas may be determined by one skilled in the art. In some embodiments, the daily dose of Aesculus turbinate may be determined by one skilled in the art. In some embodiments, the daily dose of Ceratonia siliqua pulp may be determined by one skilled in the art. In some embodiments, the daily dose of Oolong tea may be about 100 mg to about 3,000 mg. In some embodiments, the daily dose of Caralluma fimbriata may be determined by one skilled in the art. In some embodiments, the daily dose of Hoodia gordonii may be about 300 mg to about 3,000 mg. In some embodiments, the daily dose of Parasitic loranthus may be determined by one skilled in the art. In some embodiments, the daily dose of Paeonia suffruticosa may be determined by one skilled in the art. In some embodiments, the daily dose of Turnera diffusa may be determined by one skilled in the art. In some embodiments, the daily dose of Sambuca nigra may be determined by one skilled in the art. In some embodiments, the daily dose of Asparagus officinalis may be determined by one skilled in the art. In some embodiments, the daily dose of acetic acid (vinegar) may be about 15 ml. In some embodiments, the daily dose of Rosmarinus officinalis may be determined by one skilled in the art. In some embodiments, the daily dose of green coffee may be determined by one skilled in the art.

In some embodiments, the predetermined treatment phases have a pre-determined duration of about 1 week to about 20 weeks. The term “treatment phase” when used herein refers to the amount of time determined by one skilled in the art would administer a weight loss agent to cause weight loss. In some embodiments, the predetermined treatment phases have a pre-determined duration of about 4 weeks. In some embodiments, the predetermined treatment phases have a pre-determined duration of about 1, 2, 3, 4, 5, 6, 7, 8, 9, 10, 11, 12, 13, 14, 15, 16, 17, 18, 19 or 20 weeks. In some embodiments, the predetermined treatment phase is shorter than the duration required for a plateau effect of the weight loss agent. For example, where the weight loss effected by a particular weight loss agent reaches a plateau at 8 weeks, the predetermined treatment phase would be less than or equal to 8 weeks.

In some embodiments, at least two weight loss agents are administered during each treatment phase, wherein the at least one weight loss agent administered during each treatment phase is selected from a different therapeutic class than the at least one weight loss agent administered during the preceding or proceeding treatment phase.

The term “preceding” when used herein is referring to the treatment phase immediately before the current treatment phase. The term “proceeding” when used herein is referring to the treatment phase immediately after the current treatment phase.

Some embodiments are directed to a method of effecting weight loss in a subject comprising a first treatment phase, a second treatment phase and a third treatment phase.

In some embodiments the first treatment phase may comprise administering at least one weight loss agent. In some embodiments, the at least one weight loss agent is a protein kinase A modulator, a neurotransmitter modulator or a combination thereof. In some embodiments, the weight loss agents administered during this treatment phase are selected from Camellia sinensis, caffeine, Capsicum sp., 5-hydroxytryptophan, and any combination thereof. In yet other embodiments the first treatment phase comprises administering Camellia sinensis, caffeine, Capsicum sp., 5-hydroxytryptophan, or any combination thereof. Table 1 shows the estimated energy intake reduction and estimated weight loss from these agents administered individually and in combination. In some embodiments, the estimated combined weight loss with this treatment phase is about 4.5 to about 5.5 kg for a 4 week or 6 week treatment duration respectively, with an estimated energy intake reduction of about 470 Kcal/day.

TABLE 1
Estimated effect of first treatment phase

| | | Estimated | |
|---|---|---|---|
| Weight loss agent | Estimated kcal/day reduction | ΔWt. Loss_{MAX} | |
| Camellia sinensis | 120 Kcal/day | 2.0 | kg |
| caffeine | 60 Kcal/day | 0.5 | kg |
| Capsicum annum | 50 Kcal/day | 0.5 | kg |
| 5-hydroxytryptophan | 240 Kcal/day | 1.5-2.5 | kg |
| Total | 470 Kcal/day | 4.5-5.5 | kg |

In some embodiments, dosing of weight loss agents to be used in the first treatment phase may depend on the weight loss agents used. In some embodiments, the dosing of Camellia sinensis may be about once-a-day to about three-times-a-day. In some embodiments, caffeine may be administered in a single daily dose. In some embodiments, dosing of Capsicum annum may in the form of a single daily dose and in some embodiments may be administered during, or before meals. In some embodiments, dosing of 5-hydroxytryptophan may be about five times-a-day. In some embodiments, dosing of 5-hydroxytryptophan may be administered during, or before meals. In some embodiments, all of the agents in this treatment phase may be administered twice a day. In some embodiments, all of the agents in this treatment phase may be administered about once-a-day to about three-times-a-day. In some embodiments, the first treatment phase is carried out for a period of about four to about six weeks.

In some embodiments, the second treatment phase comprises administering at least one weight loss agent. In some embodiments, the at least one weight loss agent is a macro nutrient absorption modulator, a neurotransmitter modulator or a combination thereof. In some embodiments, the at least one weight loss agent is selected from Phaseolus vulgaris, Irvingia gabonensis, Rhodiola rosea, and any combination thereof. In yet other embodiments the second treatment phase comprises administering Phaseolus vulgaris, Irvingia gabonensis, Rhodiola rosea, or any combination thereof. In some embodiments, the estimated combined effect of this treatment phase is about 0.8 kg/week or about 3.2 to about 4.8 kg for a 4 week or 6 week treatment duration respectively.

TABLE 2
Estimated effect of second treatment phase

| | | | Estimated |
|---|---|---|---|
| Weight loss agent | Estimated kg/wk reduction | | ΔWt. Loss_{MAX} |
| Phaseolus vulgaris | 0.2-0.25 | kg/wk | |
| Irvingia gabonensis | 0.6 | kg/wk | |
| Rhodiola rosea | <0.1 | kg/wk | |
| Total | 0.8 | kg/wk | 3.2-4.8 kg |

In some embodiments, dosing of weight loss agents to be used in the second treatment phase may depend on the weight loss agents used. In some embodiments, dosing for Cissus quadrangularis and Irvingia gabonensis may be twice daily to three times daily and in some embodiments, may be administered before meals. In some embodiments, the dosing of Rhodiola rosea may be twice daily. In some embodiments, all of the agents in this treatment phase may be administered twice a day. In some embodiments, all of the agents in this treatment phase may be administered about once-a-day to about three-times-a-day. In some embodiments, the second treatment phase is carried out for a period of about four to about six weeks.

In some embodiments, the third treatment phase comprises administering at least one weight loss agent. In some embodiments, the at least one weight loss agent is a Protein Kinase A modulator, Adenosine Mono-Phosphate activated Kinase (AMPk) modulator, or a combination thereof. In some embodiments, the at least one weight loss agent is selected from Coffea canefora, Coleus forskholii, yohimbine, caffeine, and any combination thereof. In yet other embodiments, the third treatment phase comprises administering Coffea canefora, Coleus forskholii, yohimbine, caffeine, or any combination thereof. In some embodiments, the estimated combined effect of this treatment phase is about 0.55 kg/week for a total weight loss of 5.5 kg for a 10 week treatment period.

TABLE 3
Estimated effect of third treatment phase

| | | Estimated |
|---|---|---|
| Weight loss agent | Estimated kg/wk reduction | ΔWt. Loss_{MAX} |
| Coffea canefora | 0.3 kg/wk | 2.5 kg |
| Coleus forskholii | 0.1 kg/wk | 1.5 kg |
| yohimbine | 0.1 kg/wk | 1.5 kg |
| Caffeine | 0.05 kg/wk | 0.5 kg |
| Total | 0.55 kg/wk | 5.5 kg |

In some embodiments, the dosing regimen for the third treatment phase may depend on the weight loss agents used. In some embodiments, Coffea canefora may be administered between about twice a day and about five times a day. In some embodiments, Coleus forskholii may be administered twice a day and in some embodiments, it may administered before meals. In some embodiments, yohimbine may be administered between about twice a day and four times a day. In some embodiments, caffeine may be administered in a single daily dose. In some embodiments, all of the agents in this treatment phase may be administered twice a day. In some embodiments, all of the agents in this treatment phase may be administered about once-a-day to about three-times-a-day. In some embodiments, the third treatment phase is carried out for a period of about four to about ten weeks. In some embodiments, the third treatment phase is carried out for a period of about six to about eight weeks. In some embodiments, the third treatment phase is carried out for a period of about four to about six weeks.

In some embodiments, the method of effecting weight loss in a subject may consist of a first treatment phase, a second treatment phase, and a third treatment phase. In some embodiments, the first treatment phase of the method of effecting weight loss comprises of administering at least one weight loss agent. In some embodiments, the weight loss agent is administered about once to about three times daily. In some embodiments, the weight loss agent is administered twice daily. In some embodiments, the weight loss agent is administered twice daily for about 4 weeks to about 6 weeks. In some embodiments, the at least one weight loss agent is a protein kinase A modulator, a neurotransmitter modulator, or a combination thereof. In some embodiments, the at least one weight loss agent is selected from Camellia sinensis, caffeine, Capsicum sp., 5-hydroxytryptophan, Griffonia simplicifolia, Crocus sativus, Mucuna pruriens, L-theanine, St. John's Wort, Catha edulis, Rhodiola rosea, Quercitin, Garcina sp. and any combination thereof. In some embodiments, the amount of Camellia sinensis is about 400 mg to about 1400 mg. In some embodiments, the amount of caffeine is about 100 mg to about 1,500 mg. In some embodiments, the amount of Capsicum sp. is about 7.5 mg to about 150 mg. In some embodiments, the amount of 5-hydroxytryptophan is about 50 mg to about 900 mg. In some embodiments, the amount of Griffonia simplicifolia is about 150 mg to about 900 mg. In some embodiments, the amount of Crocus sativus is about 30 mg to about 176.5 mg. In some embodiments, the amount of Mucuna pruriens is about determined to achieve about 60 mg to about 120 mg of L-dopa. In some embodiments, the amount of L-theanine is about 200 mg to about 800 mg. In some embodiments, the amount of St. John's Wort is about 27 mg to about 300 mg. In some embodiments, the amount of Catha edulis is a therapeutically effective amount. In some embodiments, the amount of Rhodiola rosea is about 50 mg to about 680 mg. In some embodiments, the amount of Quercitin is greater than 1,000 mg. In some embodiments, the amount of Garcina sp. is about 500 mg to about 3,000 mg.

In some embodiments, the at least one weight loss agent of the first treatment phase administered is selected from Camellia sinensis, caffeine, Capsicum sp., 5-hydroxytryptophan, Griffonia simplicifolia, Crocus sativus, Mucuna pruriens, L-theanine, St. John's Wort, Catha edulis, Rhodiola rosea, Quercitin, Garcina sp. or any combination thereof. In some embodiments, the amount of Camellia sinensis is about 400 mg to about 1400 mg. In some embodiments, the amount of caffeine is about 100 mg to about 1,500 mg. In some embodiments, the amount of Capsicum sp. is about 7.5 mg to about 150 mg. In some embodiments, the amount of 5-hydroxytryptophan is about 50 mg o about 900 mg. In some embodiments, the amount of Griffonia simplicifolia is about 150 mg to about 900 mg. In some embodiments, the amount of Crocus sativus is about 30 mg to about 176.5 mg. In some embodiments, the amount of Mucuna pruriens is about determined to achieve about 60 mg to about 120 mg of L-dopa. In some embodiments, the amount of L-theanine is about 200 mg to about 800 mg. In some embodiments, the amount of St. John's Wort is about 27 mg to about 300 mg. In some embodiments, the amount of Catha edulis is a therapeutically effective amount. In some embodiments, the amount of Rhodiola rosea is about 50 mg to about 680 mg. In some embodiments, the amount of Quercitin is greater than 1,000 mg. In some embodiments, the amount of Garcina sp. is about 500 mg to about 3,000 mg. In some embodiments, the weight loss agent is administered twice daily. In some embodiments, all of the agents in this treatment phase may be administered about once-a-day to about three-times-a-day. In some embodiments, the weight loss agent is administered twice daily for about 4 to about 6 weeks.

In some embodiments, the second treatment phase of the method of effecting weight loss includes of administering at least one weight loss agent. In some embodiments, the weight loss agent is administered twice daily. In some embodiments, all of the agents in this treatment phase may be administered about once-a-day to about three-times-a-day. In some embodiments, the weight loss agent is administered twice daily for about 4 weeks to about 6 weeks. In some embodiments, the at least one weight loss agent is a macro nutrient absorption modulator, a neurotransmitter modulator or a combination thereof. In some embodiments, the at least one weight loss agent is selected from Phaseolus vulgaris, Irvingia gabonensis, Rhodiola rosea, Griffonia simplicifolia, Crocus sativus, Mucuna pruriens, L-theanine, St. John's Wort, Catha edulis, Rhodiola rosea, Quercitin, Garcina sp. and any combination thereof. In some embodiments, the amount of Phaseolus vulgaris is about 2,000 amu² to about 3,000 amu². In some embodiments, the amount of Irvingia gabonensis is about 150 mg to about 500 mg. In some embodiments, the amount of Rhodiola rosea is about 50 mg to about 680 mg. In some embodiments, the amount of Griffonia simplicifolia is about 150 mg to about 900 mg. In some embodiments, the amount of Crocus sativus is about 30 mg to about 176.5 mg. In some embodiments, the amount of Mucuna pruriens is about determined to achieve about 60 mg to about 120 mg of L-dopa. In some embodiments, the amount of L-theanine is about 200 mg to about 800 mg. In some embodiments, the amount of St. John's Wort is about 27 mg to about 300 mg. In some embodiments, the amount of Catha edulis is a therapeutically effective amount. In some embodiments, the amount of Rhodiola rosea is about 50 mg to about 680 mg. In some embodiments, the amount of Quercitin is greater than 1,000 mg. In some embodiments, the amount of Garcina sp. is about 500 mg to about 3,000 mg.

In some embodiments, the at least one weight loss agent of the second treatment phase administered is selected from Phaseolus vulgaris, Irvingia gabonensis, Rhodiola rosea, Griffonia simplicifolia, Crocus sativus, Mucuna pruriens, L-theanine, St. John's Wort, Catha edulis, Rhodiola rosea, Quercitin, Garcina sp. or a combination thereof. In some embodiments, the weight loss agent is administered twice daily. In some embodiments, all of the agents in this treatment phase may be administered about once-a-day to about three-times-a-day. In some embodiments, the weight loss agent is administered twice daily for about 4 to about 6 weeks. In some embodiments, the amount of Phaseolus vulgaris is about 2,000 amu² to about 3,000 amu². In some embodiments, the amount of Irvingia gabonensis is about 150 mg to about 500 mg. In some embodiments, the amount of Rhodiola rosea is about 50 mg to about 680 mg. In some embodiments, the amount of Griffonia simplicifolia is about 150 mg to about 900 mg. In some embodiments, the amount of Crocus sativus is about 30 mg to about 176.5 mg. In some embodiments, the amount of Mucuna pruriens is about determined to achieve about 60 mg to about 120 mg of L-dopa. In some embodiments, the amount of L-theanine is about 200 mg to about 800 mg. In some embodiments, the amount of St. John's Wort is about 27 mg to about 300 mg. In some embodiments, the amount of Catha edulis is a therapeutically effective amount. In some embodiments, the amount of Rhodiola rosea is about 50 mg to about 680 mg. In some embodiments, the amount of Quercitin is greater than 1,000 mg. In some embodiments, the amount of Garcina sp. is about 500 mg to about 3,000 mg.

In some embodiments, the third treatment phase of the method of effecting weight loss includes of administering at least one weight loss agent. In some embodiments, the weight loss agent is administered twice daily. In some embodiments, all of the agents in this treatment phase may be administered about once-a-day to about three-times-a-day. In some embodiments, the weight loss agent is administered twice daily for about 4 weeks to about 10 weeks. In some embodiments, the weight loss agent is administered twice daily for about 6 weeks to about 8 weeks. In some embodiments, the weight loss agent is administered twice daily for about 4 weeks to about 6 weeks. In some embodiments, the at least one weight loss agent is a protein kinase A modulator, an adenosine mono-phosphate activated kinase modulator or a combination thereof. In some embodiments, the at least one weight loss agent is selected from Coffea canefora, Coleus forskohlii, yohimbine, caffeine, Salix alba, Ilex paraguariensis, Ecklonia cava, phillyrin, Rubus idaeus, Panax ginseng berry and root and any combination thereof. In some embodiments, the amount of Coffea canefora is about 200 mg to about 1600 mg. In some embodiments, the amount of Coleus forskohlii is about 25 mg to about 75 mg of forskolin. In some embodiments, the amount of yohimbine is about 7.5 mg to about 1,000 mg. In some embodiments, the amount of caffeine is about 100 mg to about 1,500 mg. In some embodiments, the amount of Salix alba is an amount equivalent to about 15 mg to about 120 mg of total salicin. In some embodiments, the amount of Ilex paraguariensis is about 100 mg to about 3,000 mg. In some embodiments, the daily dose of Ecklonia cava may be administered at a dose greater than 1.6 mg. In some embodiments, the amount of phillyrin is a therapeutically effective amount. In some embodiments, the amount of Rubus idaeus is a therapeutically effective amount. In some embodiments, the amount of Panax ginseng berry and root is about 1,500 mg to about 20,000 mg.

In some embodiments, the at least one weight loss agent of the third treatment phase administered is selected from Coffea canefora, Coleus forskohlii, yohimbine, caffeine, Salix alba, Ilex paraguariensis, Ecklonia cava, phillyrin, Rubus idaeus, Panax ginseng berry and root or a combination thereof. In some embodiments, the weight loss agent is administered twice daily. In some embodiments, all of the agents in this treatment phase may be administered about once-a-day to about three-times-a-day. In some embodiments, the weight loss agent is administered twice daily for about 4 to about 10 weeks. In some embodiments, the weight loss agent is administered twice daily for about 6 weeks to about 8 weeks. In some embodiments, the weight loss agent is administered twice daily for about 4 weeks to about 6 weeks. In some embodiments, the amount of Coffea canefora is about 200 mg to about 1600 mg. In some embodiments, the amount of Coleus forskohlii is about 25 mg to about 75 mg of forskolin. In some embodiments, the amount of yohimbine is about 7.5 mg to about 1,000 mg. In some embodiments, the amount of caffeine is about 100 mg to about 1,500 mg. In some embodiments, the amount of Salix alba is an amount equivalent to about 15 mg to about 120 mg of total salicin. In some embodiments, the amount of Ilex paraguariensis is about 100 mg to about 3,000 mg. In some embodiments, the daily dose of Ecklonia cava may be administered at a dose greater than 1.6 mg. In some embodiments, the amount of phillyrin is a therapeutically effective amount. In some embodiments, the amount of Rubus idaeus is a therapeutically effective amount. In some embodiments, the amount of Panax ginseng berry and root is about 1,500 mg to about 20,000 mg.

Any of the compositions or kits described herein can be used to prolong weight loss. As described herein, most weight loss agents lose effectiveness anywhere from 4 to 10 weeks after initiation of a weight loss program. This occurs once the body has compensated to the weight loss agent effect.

Methods of Prolonging the Effect of Weight Loss Agents

Some embodiments are directed to methods of prolonging the effect of a weight loss agent in a subject comprising: defining at least two treatment phases of pre-determined duration; administering at least one weight loss agent during each treatment phase; wherein the at least one weight loss agent administered during each treatment phase is selected from a different therapeutic class than the at least one weight loss agent administered during the preceding or proceeding treatment phase.

In some embodiments, the methods of prolonging weight loss result in weight loss over a greater period of time than when weight loss agents are used individually. In some embodiments, prolonging weight loss results in a delay or in the disappearance of the weight loss plateau.

In some embodiments, the therapeutic class is selected from neurotransmitter modulators, hormonal satiety or hunger signaling modulators, β-adrenergic receptor modulators, α2-adrenergic modulators, adenosine receptor modulators, cannabinoid receptor modulators, protein-tyrosine phosphatase 1B modulators, catechol O-methyltransferase modulators, AMPk modulators, phosphodiesterase modulators, non-guanosine nucleotide mediated adenylate cyclase modulators, hormone modulators, glucose metabolism modulators, carbohydrate metabolism modulators, lipid metabolism modulators, protein kinase A and agents with an unknown mechanism of action.

In some embodiments, neurotransmitter modulators are selected from but are not limited to Griffonia simplicifolia, Crocus sativus, Mucuna pruriens, L-theanine, St. John's Wort, Catha edulis, Rhodiola rosea, Quercitin, and Garcina sp. In some embodiments, hormonal satiety or hunger signaling modulators are selected from but are not limited to Slendesta™, Fenugreek (Trigonella foenum-graecum), Plantago ovata (Plantago psyllium), Fibersol® 2, alginate, conjugated linoleic acid, α-lipoic acid, diacylglycerol, linolenate, Cocos nucifera oleum, whey protein, soy protein, Coix lacryma-jobi, Ilex paraguariensis, Quercitin, Agave tequilana, Dasylirion sp., and Panax ginseng. In some embodiments, β-adrenergic receptor modulators are selected from but are not limited to Citrus aurantium and Capsicum sp., also known as capsinoids. In some embodiments, α₂-adrenergic receptor modulators are selected from but are not limited to yohimbine and Rubus idaeus. In some embodiments, adenosine receptor modulators are selected from but are not limited to caffeine, Guarana sp., Ilex paraguariensis, xanthine alkaloids, and theobromine. In some embodiments, cannabinoid receptor modulators are selected from but are not limited to PhosphoLean®. In some embodiments, protein-tyrosine phosphatase 1B (PTP1B) modulators are selected from but are not limited to Cinnamomum cassia. In some embodiments, catechol O-methyltransferase (COMT) modulators are selected from but are not limited to Camellia sinensis and PhosphoLean®. In some embodiments, 5′ AMP-activated protein kinase (AMPk) modulators are selected from but are not limited to Salix alba, Coffea canefora, Ilex paraguariensis, Ecklonia cava, phillyrin, Rubus idaeus, and Panax ginseng berry and root. In some embodiments, phosphodiesterase modulators are selected from but are not limited to Sinetrol®, phillyrin, caffeine, Guarana sp., Ilex paraguariensis and theobromine. In some embodiments, non-guanosine nucleotide mediated adenylate cyclase modulators, also known as Non-G-protein adenylate cyclase modulators, are selected from but are not limited to Coleus forskholii. In some embodiments, glucose metabolism modulators are selected from but are not limited to Momordica charantia, Olea europea and Theobroma cacao bean. In some embodiments, carbohydrate metabolism modulators are selected from but are not limited to Garcinia sp., Coffea canefora, Phaseolus vulgaris, Cissus quadrangularis, Irvingia gabonensis, Carthamus tinctorius, Laminaria japonica, Curcuma longa, Zingiber officinale, Rosemarinus officinalis and Gymnema sylvestre. In some embodiments, lipid metabolism modulators are selected from but are not limited to Isohumulones, also known as Humulus lupulus, Arachis hypogaea, Allium victorialis, Punica granatum leaf, Kochis scoparia, Panax japonicas, Aesculus turbinate, Ceratonia siliqua pulp, oolong tea, Ilex paraguariensis, Glycyrrhiza glabra, and Cissus quadrangularis. In some embodiments, agents with an unknown mechanism of action are selected from but are not limited to Caralluma fimbriata, Hoodia gordonii, Parasitic loranthus, Paeonia suffruticosa, Turnera diffusa, Sambuca nigra, Asparagus officinalis and acetic acid (vinegar). In some embodiments, the predetermined treatment phases have a pre-determined duration of about 1 week to about 20 weeks. In some embodiments, the predetermined treatment phases have a pre-determined duration of about 4 weeks.

In some embodiments, at least two weight loss agents are administered during each treatment phase, wherein the at least one weight loss agent administered during each treatment phase is selected from a different therapeutic class than the at least one weight loss agent administered during the preceding or proceeding treatment phase.

In some embodiments, the predetermined treatment phase is shorter than the duration required for a plateau effect of the weight loss agent. For example, where the weight loss effected by a particular weight loss agent reaches a plateau at 8 weeks, the predetermined treatment phase would be less than or equal to 8 weeks.

Kits for Effecting Weight Loss

Some embodiments are directed to a kit for effecting weight loss in a subject comprising: at least two weight loss agents; and instructions for defining at least two treatment phases of pre-determined duration and administering at least one weight loss agent during each treatment phase; wherein the at least one weight loss agent administered during each treatment phase is selected from a different therapeutic class than the at least one weight loss agent administered during the preceding or proceeding treatment phase.

The kit provides a suitable format for the convenient and accurate dosing and distribution of weight loss agents according to a predetermined treatment phase. Examples of suitable forms include, but are not limited to, blister packs, bottles, ampules, patches.

In some embodiments, the weight loss agents to be administered during a single treatment phase can be combined into a weight loss composition. In some embodiments, the weight loss agents to be administered in a single treatment phase are to be administered as separate weight loss compositions comprising a single weight loss agent.

In some embodiments, the kit may include instructions for administering the at least two weight loss agents and for defining at least two treatment phases of pre-determined duration and administering at least one weight loss agent during each treatment phase. For example, in some embodiments, the instructions may direct a subject to administer a first of the at least two weight loss agents for a first of at least two treatment phases of pre-determined duration of 4 weeks and then administering the second the at least two weight loss agents for a second of at least two treatment phases of pre-determined duration of 4 weeks.

In some embodiments, the kits embodied herein may comprise sufficient individual doses of the at least two weight loss agents to administer the at least two weight loss agents for the least two treatment phases of pre-determined duration. In some embodiments, the weight loss agents may be configured for daily administration. In some embodiments, the individual dose of the weight loss agent is configured to comprise a single daily dose. In some embodiments, the individual doses of a weight loss agent comprise a fraction of the daily dose such as but not limited half the daily dose for administration twice a day or a third of the daily dose for administration three times a day. In yet other embodiments, the individual doses can be configured in any suitable combination to provide the desired daily dose of a weight loss agent.

In some embodiments, the predetermined treatment phase is shorter than the duration required for a plateau effect of the weight loss agent. For example, where the weight loss effected by a particular weight loss agent reaches a plateau at 8 weeks, the predetermined treatment phase would be less than or equal to 8 weeks.

In some embodiments, the therapeutic class is selected from neurotransmitter modulators, hormonal satiety or hunger signaling modulators, β-adrenergic receptor modulators, α2-adrenergic modulators, adenosine receptor modulators, cannabinoid receptor modulators, protein-tyrosine phosphatase 1B modulators, catechol O-methyltransferase modulators, AMPk modulators, phosphodiesterase modulators, non-guanosine nucleotide mediated adenylate cyclase modulators, glucose metabolism modulators, carbohydrate metabolism modulators, lipid metabolism modulators, protein kinase A modulators and agents with an unknown mechanism of action.

In some embodiments, neurotransmitter modulators are selected from but are not limited to Griffonia simplicifolia, Crocus sativus, Mucuna pruriens, L-theanine, St. John's Wort, Catha edulis, Rhodiola rosea, Quercitin, and Garcina sp. In some embodiments, hormonal satiety or hunger signaling modulators are selected from but are not limited to Slendesta™, Fenugreek (Trigonella foenum-graecum), Plantago ovata (Plantago psyllium), Fibersol® 2, alginate, conjugated linoleic acid, α-lipoic acid, diacylglycerol, linolenate, Cocos nucifera oleum, whey protein, soy protein, Coix lacryma-jobi, Ilex paraguariensis, Quercitin, Agave tequilana, Dasylirion sp., and Panax ginseng. In some embodiments, β-adrenergic receptor modulators are selected from but are not limited to Citrus aurantium and Capsicum sp., also known as capsinoids. In some embodiments, α₂-adrenergic receptor modulators are selected from but are not limited to yohimbine and Rubus idaeus. In some embodiments, adenosine receptor modulators are selected from but are not limited to caffeine, Guarana sp., Ilex paraguariensis, xanthine alkaloids, and theobromine. In some embodiments, cannabinoid receptor modulators are selected from but are not limited to PhosphoLean®. In some embodiments, protein-tyrosine phosphatase 1B (PTP1B) modulators are selected from but are not limited to Cinnamomum cassia. In some embodiments, catechol O-methyltransferase (COMT) modulators are selected from but are not limited to Camellia sinensis and PhosphoLean®. In some embodiments, 5′ AMP-activated protein kinase (AMPk) modulators are selected from but are not limited to Salix alba, Coffea canefora, Ilex paraguariensis, Ecklonia cava, phillyrin, Rubus idaeus, and Panax ginseng berry and root. In some embodiments, phosphodiesterase modulators are selected from but are not limited to Sinetrol®, phillyrin, caffeine, Guarana sp., Ilex paraguariensis and theobromine. In some embodiments, non-guanosine nucleotide mediated adenylate cyclase modulators, also known as Non-G-protein adenylate cyclase modulators, are selected from but are not limited to Coleus forskholii. In some embodiments, glucose metabolism modulators are selected from but are not limited to Momordica charantia, Olea europea and Theobroma cacao bean. In some embodiments, carbohydrate metabolism modulators are selected from but are not limited to Garcinia sp., Coffea canefora, Phaseolus vulgaris, Cissus quadrangularis, Irvingia gabonensis, Carthamus tinctorius, Laminaria japonica, Curcuma longa, Zingiber officinale, Rosemarinus officinalis and Gymnema sylvestre. In some embodiments, lipid metabolism modulators are selected from but are not limited to Isohumulones, also known as Humulus lupulus, Arachis hypogaea, Allium victorialis, Punica granatum leaf, Kochis scoparia, Panax japonicas, Aesculus turbinate, Ceratonia siliqua pulp, oolong tea, Ilex paraguariensis, Glycyrrhiza glabra, and Cissus quadrangularis. In some embodiments, agents with an unknown mechanism of action are selected from but are not limited to Caralluma fimbriata, Hoodia gordonii, Parasitic loranthus, Paeonia suffruticosa, Turnera diffusa, Sambuca nigra, Asparagus officinalis and acetic acid (vinegar).

In some embodiments, the predetermined treatment phases have a pre-determined duration of about 1 week to about 20 weeks. In some embodiments, the predetermined treatment phases have a pre-determined duration of about 4 weeks.

In some embodiments, at least two weight loss agents are administered during each treatment phase, wherein the at least one weight loss agent administered during each treatment phase is selected from a different therapeutic class than the at least one weight loss agent administered during the preceding or proceeding treatment phase.

In some embodiments, the predetermined treatment phase is shorter than the duration required for a plateau effect of the weight loss agent. For example, where the weight loss effected by a particular weight loss agent reaches a plateau at 8 weeks, the predetermined treatment phase would be less than or equal to 8 weeks.

In some embodiments, the weight loss agents, weight loss compositions or combinations thereof may include a pharmaceutical excipient. In some embodiments, the pharmaceutical excipient may include, without limitation, binders, coating, disintegrants, fillers, diluents, flavors, colors, lubricants, glidants, preservatives, sorbents, sweeteners, conjugated linoleic acid (CLA), gelatin, any type of oil, including, without limitation, fish oil or soybean oil, or the like. In some embodiments, the nutritional supplement composition may include an enteric coating.

In some embodiments, the weight loss agents, weight loss compositions, or combinations thereof may further comprise other weight loss agents, such as, for example, other vitamins, herbs, hormones, minerals or essential fatty acids, to promote cardiovascular, heart, bone, or liver health. For example, in some embodiments, the nutritional supplement may further comprise acai, calcium, biotin, folic acid, vitamin A, curcumin, vitamin B12, piperine, fish oil, carotene, magnesium, potassium, zinc, lipase, amylase, or the like.

The weight loss agents, weight loss compositions, or combinations thereof of the present invention can be administered in the conventional manner by any route where they are active. Administration can be systemic, parenteral, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration may depend on the indication. The selection of the specific route of administration and the dose regimen is to be adjusted or titrated by one skilled in the art according to methods known to one skilled in the art in order to obtain the optimal response. The amount of compound to be administered is that amount which is therapeutically effective. The dosage to be administered may depend on the characteristics of the subject being treated, e.g., the particular subject treated, age, weight, health, types of concurrent treatment, if any, and frequency of treatments, and can be easily determined by one of skill in the art (e.g., by the one skilled in the art

Weight loss agents and weight loss compositions containing the weight loss agents of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active weight loss agents can be contained in such formulations with pharmaceutically or nutraceutically acceptable diluents, fillers, disintegrants, binders, lubricants, surfactants, hydrophobic vehicles, water-soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The weight loss agents, weight loss compositions, or combinations thereof of the present invention can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the weight loss agents, weight loss compositions, or combinations thereof can be formulated readily by combining these weight loss agents with pharmaceutically or nutraceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

weight loss agents, weight loss compositions, or combinations thereof which can be used orally include, but are not limited to, push-fit capsules made of gelatin, as well as soft, sealed capsules made of gelatin and a plasticizer, such as glycerol or sorbitol. The push-fit capsules can contain the active weight loss agents in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compositions of the present invention can also be formulated in rectal compositions such as suppositories or retention enemas, e.g., containing conventional suppository bases such as cocoa butter or other glycerides.

In addition to the formulations described previously, the compositions of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present invention, for example, can be applied to a plaster, or can be applied by transdermal, therapeutic systems that are consequently supplied to the organism.

weight loss agents, weight loss compositions, or combinations thereof also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions of the present invention can also be administered in combination with other active weight loss agents, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated Pharmaceutical formulations containing the weight loss agents of the invention and a suitable carrier can be in various forms including, but not limited to, solids, solutions, powders, fluid emulsions, fluid suspensions, semi-solids, and dry powders including an effective amount of an activated fatty acid of the invention. It is also known in the art that the active weight loss agents can be contained in such formulations with pharmaceutically or nutraceutically acceptable diluents, fillers, disintegrants, binders, lubricants, surfactants, hydrophobic vehicles, water soluble vehicles, emulsifiers, buffers, humectants, moisturizers, solubilizers, antioxidants, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's, The Pharmaceutical Basis of Therapeutics, 6th Edition, MacMillan Publishing Co., New York (1980) both of which are hereby incorporated by reference in their entireties can be consulted.

The weight loss agents, weight loss compositions, or combinations thereof of the present invention can be formulated for parenteral or intravenous administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids diluents such as oleic acid find use in the preparation of injectables. Additional fatty acids diluents that may be useful in embodiments of the invention include, for example, one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, and the like. In some embodiments, the fatty acid diluent may be a mixture of fatty acids. In some embodiments, the fatty acid may be a fatty acid ester, a sugar ester of fatty acid, a glyceride of fatty acid, or an ethoxylated fatty acid ester, and in other embodiments, the fatty acid diluent may be a fatty alcohol such as, for example, stearyl alcohol, lauryl alcohol, palmityl alcohol, palmitolyl acid, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol and the like and mixtures thereof.

Other embodiments of the invention include weight loss agents, weight loss compositions, or combinations thereof formulated as a solid dosage form for oral administration including capsules, tablets, pills, powders, and granules. In such embodiments, the weight loss agent may be admixed with one or more inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents and can additionally be prepared with enteric coatings.

Preparation of a weight loss agents, weight loss compositions, or combinations thereof in solid dosage form may vary. For example, in one embodiment, a liquid or gelatin formulation of the activated fatty acid may be prepared by combining the activated fatty acid with one or more fatty acid diluent, such as those described above, and adding a thickening agent to the liquid mixture to form a gelatin. The gelatin may then be encapsulated in unit dosage form to form a capsule. In another exemplary embodiment, an oily preparation of a weight loss agent above may be lyophilized to for a solid that may be mixed with one or more pharmaceutically or nutraceutically acceptable excipient, carrier or diluent to form a tablet, and in yet another embodiment, the weight loss agent an oily preparation may be crystallized to from a solid which may be combined with a pharmaceutically or nutraceutically acceptable excipient, carrier or diluent to form a tablet.

Further embodiments which may be useful for oral administration of weight loss agents include liquid dosage forms. In such embodiments, a liquid dosage may include a pharmaceutically or nutraceutically acceptable emulsion, solution, suspension, syrup, and elixir containing inert diluents commonly used in the art, such as water. Such compositions may also comprise adjuvants, such as wetting agents, emulsifying and suspending agents, and sweetening, flavoring, and perfuming agents.

In still further embodiments, weight loss agents, weight loss compositions, or combinations thereof of the invention can be formulated as a depot preparation. Such long acting formulations can be administered by implantation (for example, subcutaneously or intramuscularly) or by intramuscular injection. Depot injections can be administered at about 1 to about 6 months or longer intervals. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Other suitable diluents for injectable formulations include, but are not limited to those described below:

Vegetable oil: As used herein, the term “vegetable oil” refers to a compound, or mixture of compounds, formed from ethoxylation of vegetable oil, wherein at least one chain of polyethylene glycol is covalently bound to the vegetable oil. In some embodiments, the fatty acids has between about twelve carbons to about eighteen carbons. In some embodiments, the amount of ethoxylation can vary about 2 to about 200, about 5 to 100, about 10 to about 80, about 20 to about 60, or about 12 to about 18 of ethylene glycol repeat units. The vegetable oil may be hydrogenated or unhydrogenated. Suitable vegetable oils include, but are not limited to castor oil, hydrogenated castor oil, sesame oil, corn oil, peanut oil, olive oil, sunflower oil, safflower oil, soybean oil, benzyl benzoate, sesame oil, cottonseed oil, and palm oil. Other suitable vegetable oils include commercially available synthetic oils such as, but not limited to, Miglyol™ 810 and 812 (available from Dynamit Nobel Chemicals, Sweden) Neobee™ M5 (available from Drew Chemical Corp.), Alofine™ (available from Jarchem Industries), the Lubritab™ series (available from JRS Pharma), the Sterotex™ (available from Abitec Corp.), Softisan™ 154 (available from Sasol), Croduret™ (available from Croda), Fancol™ (available from the Fanning Corp.), Cutina™ HR (available from Cognis), Simulsol™ (available from CJ Petrow), EmCon™ CO (available from Amisol Co.), Lipvol™ CO, SES, and HS-K (available from Lipo), and Sterotex™ HM (available from Abitec Corp.). Other suitable vegetable oils, including sesame, castor, corn, and cottonseed oils, include those listed in R. C. Rowe and P. J. Shesky, Handbook of Pharmaceutical Excipients, (2006), 5th ed., which is incorporated herein by reference in its entirety. Suitable polyethoxylated vegetable oils, include but are not limited to, Cremaphor™ EL or RH series (available from BASF), Emulphor™ EL-719 (available from Stepan products), and Emulphor™ EL-620P (available from GAF).

Mineral oils: As used herein, the term “mineral oil” refers to both unrefined and refined (light) mineral oil. Suitable mineral oils include, but are not limited to, the Avatech™ grades (available from Avatar Corp.), Drakeol™ grades (available from Penreco), Sirius™ grades (available from Shell), and the Citation™ grades (available from Avater Corp.).

Castor oils: As used herein, the term “castor oil”, refers to a compound formed from the ethoxylation of castor oil, wherein at least one chain of polyethylene glycol is covalently bound to the castor oil. The castor oil may be hydrogenated or unhydrogenated. Synonyms for polyethoxylated castor oil include, but are not limited to polyoxyl castor oil, hydrogenated polyoxyl castor oil, mcrogolglyceroli ricinoleas, macrogolglyceroli hydroxystearas, polyoxyl 35 castor oil, and polyoxyl 40 hydrogenated castor oil. Suitable polyethoxylated castor oils include, but are not limited to, the Nikkol™ HCO series (available from Nikko Chemicals Co. Ltd.), such as Nikkol HCO-30, HC-40, HC-50, and HC-60 (polyethylene glycol-30 hydrogenated castor oil, polyethylene glycol-40 hydrogenated castor oil, polyethylene glycol-50 hydrogenated castor oil, and polyethylene glycol-60 hydrogenated castor oil, Emulphor™ EL-719 (castor oil 40 mole-ethoxylate, available from Stepan Products), the Cremophore™ series (available from BASF), which includes Cremophore RH40, RH60, and EL35 (polyethylene glycol-40 hydrogenated castor oil, polyethylene glycol-60 hydrogenated castor oil, and polyethylene glycol-35 hydrogenated castor oil, respectively), and the Emulgin® RO and HRE series (available from Cognis PharmaLine). Other suitable polyoxyethylene castor oil derivatives include those listed in R. C. Rowe and P. J. Shesky, Handbook of Pharmaceutical Excipients, (2006), 5th ed., which is incorporated herein by reference in its entirety.

Sterol: As used herein, the term “sterol” refers to a compound, or mixture of compounds, derived from the ethoxylation of sterol molecule. Suitable polyethoyxlated sterols include, but are not limited to, PEG-24 cholesterol ether, Solulan™ C-24 (available from Amerchol); PEG-30 cholestanol, Nikkol™ DHC (available from Nikko); Phytosterol, GENEROL™ series (available from Henkel); PEG-25 phyto sterol, Nikkol™ BPSH-25 (available from Nikko); PEG-5 soya sterol, Nikkol™ BPS-5 (available from Nikko); PEG-10 soya sterol, Nikkol™ BPS-10 (available from Nikko); PEG-20 soya sterol, Nikkol™ BPS-20 (available from Nikko); and PEG-30 soya sterol, Nikkol™ BPS-30 (available from Nikko). As used herein, the term “PEG” refers to polyethylene glycol.

Polyethylene glycol: As used herein, the term “polyethylene glycol” or “PEG” refers to a polymer containing ethylene glycol monomer units of formula —O—CH₂—CH₂—. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is about 200 to about 9000. In some embodiments, the average molecular weight of the polyethylene glycol is about 200 to about 5000. In some embodiments, the average molecular weight of the polyethylene glycol is about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer. In some embodiments, the polyethylene glycol is polyethylene glycol-400. Suitable polyethylene glycols include, but are not limited to the Carbowax™ and Carbowax™ Sentry series (available from Dow), the Lipoxol™ series (available from Brenntag), the Lutrol™ series (available from BASF), and the Pluriol™ series (available from BASF).

Propylene glycol fatty acid ester: As used herein, the term “propylene glycol fatty acid ester” refers to an monoether or diester, or mixtures thereof, formed between propylene glycol or polypropylene glycol and a fatty acid. Fatty acids that are useful for deriving propylene glycol fatty alcohol ethers include, but are not limited to, those defined herein. In some embodiments, the monoester or diester is derived from propylene glycol. In some embodiments, the monoester or diester has about 1 to about 200 oxypropylene units. In some embodiments, the polypropylene glycol portion of the molecule has about 2 to about 100 oxypropylene units. In some embodiments, the monoester or diester has about 4 to about 50 oxypropylene units. In some embodiments, the monoester or diester has about 4 to about 30 oxypropylene units. Suitable propylene glycol fatty acid esters include, but are not limited to, propylene glycol laurates: Lauroglycol™ FCC and 90 (available from Gattefosse); propylene glycol caprylates: Capryol™ PGMC and 90 (available from Gatefosse); and propylene glycol dicaprylocaprates: Labrafac™ PG (available from Gatefosse).

Stearoyl macrogol glyceride: Stearoyl macrogol glyceride refers to a polyglycolized glyceride synthesized predominately from stearic acid or from compounds derived predominately from stearic acid, although other fatty acids or compounds derived from other fatty acids may be used in the synthesis as well. Suitable stearoyl macrogol glycerides include, but are not limited to, Gelucire® 50/13 (available from Gattefossé).

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

Exemplary excipients or carriers for use in solid and/or liquid dosage forms include, but are not limited to:

Sorbitol: Suitable sorbitols include, but are not limited to, PharmSorbidex E420 (available from Cargill), Liponic 70-NC and 76-NC (available from Lipo Chemical), Neosorb (available from Roquette), Partech SI (available from Merck), and Sorbogem (available from SPI Polyols).

Starch, sodium starch glycolate, and pregelatinized starch include, but are not limited to, those described in R. C. Rowe and P. J. Shesky, Handbook of Pharmaceutical Excipients, (2006), 5th ed., which is incorporated herein by reference in its entirety.

Disintegrant: The disintegrant may include one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

Still further embodiments of the invention include weight loss agents administered in combination with other active ingredients such as, for example, adjuvants, protease inhibitors, or other compatible drugs or compounds where such combination is seen to be desirable or advantageous in achieving the desired effects of the methods described herein.

This invention and embodiments illustrating the method and materials used may be further understood by reference to the following non-limiting examples.

### EXAMPLES

TABLE 4
Methods of effecting weight loss

| | | |
|---|---|---|
| Therapeutic class | Weight loss agent | Dose |

EXAMPLE 1

| | | | |
|---|---|---|---|
| Treatment Phase 1 - | Neurotransmitter | Griffonia | 150 mg-900 mg |
| 4 weeks | modulator | simplicafolia | |
| Treatment Phase 2- | COMT modulator | Camellia sinensis | 400 mg-1400 mg |
| 4 weeks | | | |
| Treatment Phase 3- | Carbohydrate | Cissus | 200 mg-600 mg |
| 4 weeks | metabolism modulator, | quadrangularis | |
| | lipid metabolism | | |
| | modulator | | |
| Treatment Phase 4- | adenosine receptor | Guarana sp. | Less than 150 mg |
| 4 weeks | modulator, | (Paullinia cupana) | caffeine content |
| | phosphodiesterase | | |
| | modulator | | |

EXAMPLE 2

| | | | |
|---|---|---|---|
| Treatment Phase 1 -6 | Carbohydrate | Irvingia gabonensis | 150 mg-500 mg |
| weeks | metabolism modulator | | |
| Treatment Phase 2-6 | AMPk modulator | Ecklonia cava | Greater than 1.6 mg |
| weeks | | | |
| Treatment Phase 3-6 | non guanosine | Coleus forskohlii | 25 mg-75 mg of |
| weeks | nucleotide mediated | | forskolin |
| | adenylate cyclase | | |
| | inhibitor | | |
| Treatment Phase 4-6 | α₂ adrenergic receptor | Rubus idaeus | therapeutically |
| weeks | modulator | | effective amount |
| Treatment Phase 5-6 | Carbohydrate | Irvingia gabonensis | 150 mg-500 mg |
| weeks | metabolism modulator | | |

EXAMPLE 3

| | | | |
|---|---|---|---|
| Treatment Phase 1-8 | Agent #1 - | Agent #1 - Griffonia | 150 mg-900 mg |
| weeks | neurotransmitter | simplicafolia | 400 mg-1400 mg |
| | modulator | Agent #2 - Camellia | |
| | Agent #2 COMT | sinensis | |
| | modulator | | |
| Treatment Phase 2-8 | Agent #1 - α₂ | Agent #1 - | 7.5 mg-1000 mg |
| weeks | adrenergic receptor | yohimbine | 150 mg-500 mg |
| | modulator | Agent #2 - Irvingia | |
| | Agent #2 - | gabonensis | |
| | Carbohydrate | | |
| | metabolism modulator, | | |
| Treatment Phase 3-8 | Agent #1 - adenosine | Agent #1 - Guarana | Less than 150 mg |
| weeks | receptor modulator, | sp. (Paullinia | caffeine content |
| | phosphodiesterase | cupana) | therapeutically |
| | modulator | Agent #2 - Arachis | effective amount |
| | Agent #2 - Lipid | hypogaea | |
| | metabolism modulator | | |
| Treatment Phase 4-8 | Agent #1 -β- | Agent #1 - Citrus | 10 mg-70 mg |
| weeks | adrenergic modulator | aurantium | 27 mg-300 mg |
| | Agent #2 | Agent #2 - | |
| | neurotransmitter | St. John's | |
| | modulator | Wort | |

### Example 4

### Pilot Study

Efficacy Benchmarks

In order to better understand the underlying dynamics of weight loss agents over time, several weight loss agents were selected to study and attempt to model. These included a wide range of weight loss agents including dietary supplements (C. sinensis & alginate), OTC drugs (orlistat), and Rx drugs (sibutramine, Qysmia®). Over time, more may be added to the database to test hypotheses and to improve the modeling of the underlying weight loss dynamics.

Key Findings

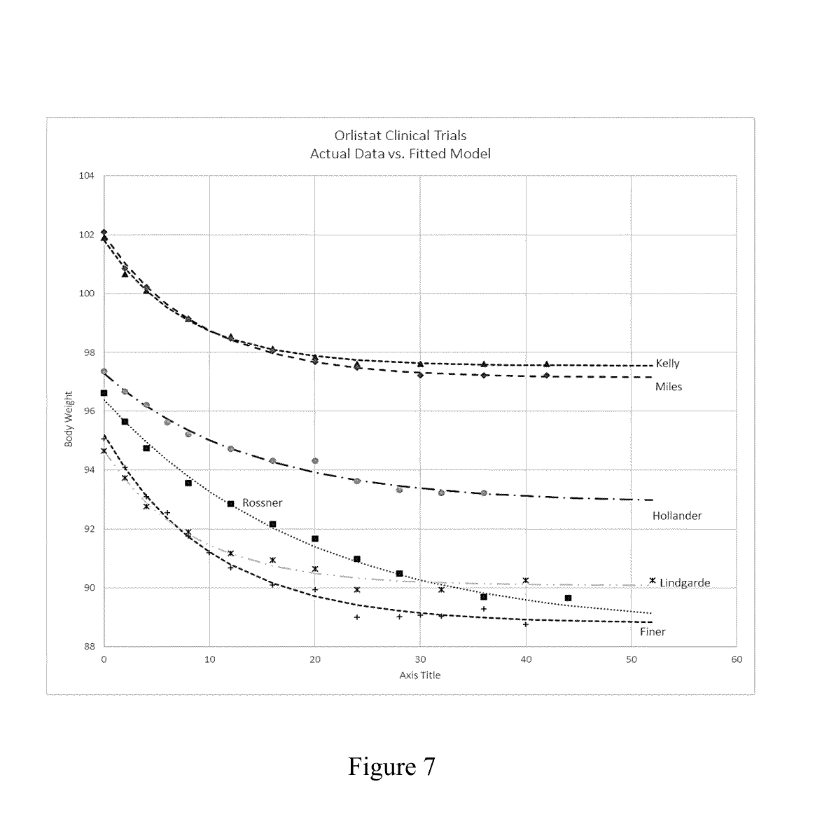

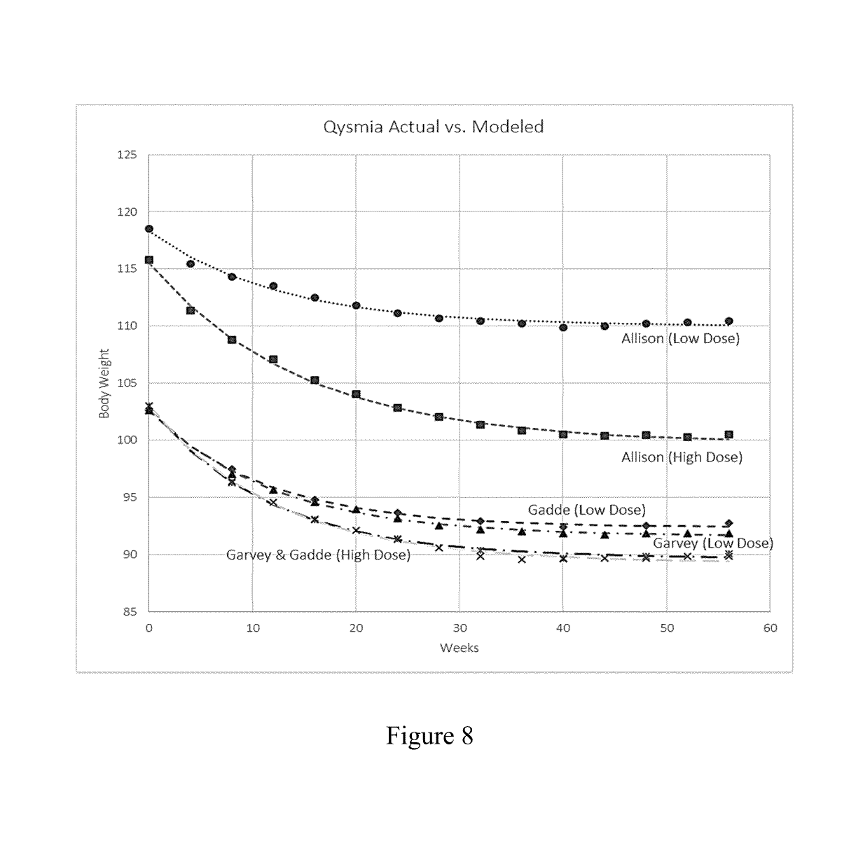

All agents followed the same basic kinetic model: Weight (t)=Weight Plateau+ΔWt. Loss_{MAX} e-^{bt}, where Weight Plateau=Starting Weight−ΔWt. Loss_{MAX}. Where t=time, b=decay coefficient, and ΔWt. Loss_{MAX}=maximum amount of weight lost, which appears to be a function of weight loss agent, dosage, and patient population. The decay curve coefficient does not appear to be linked to dose or patient population. It does appear to vary by weight loss agent. The variation within a weight loss agent appears to be fairly low. It is critical to note that these are only the initial observations based upon estimating the results using published data. The raw data, more observations, and a more sophisticated statistical analysis are needed in order to make more definitive claims. The fact that all weight loss agents seem to follow the same relationship supports the adaptation hypothesis. FIG. 7 shows the results for orlistat. All of the observed data fit the model well. The decay coefficient was estimated to be 0.1±0.02, and all the studies except Rossner fell within one standard deviation of this mean. The decay coefficient for the Rossner study was 0.05, which is slightly more than two standard deviations from the mean. However, it is important to put this in context. The maximum difference between the model using 0.05 as the decay coefficient and 0.10 is about 2.0 kg, which falls within the observation error range. It is unclear whether this difference was due to random chance, to variation caused by the inherent error arising from estimating the data from the published graphs, or to something more fundamental. Consistent with the results presented by Hutton et al., a significant difference was seen in the ΔWt. Loss_{MAX} for diabetic vs non-diabetic patients on orlistat. No difference was seen in the decay coefficient between these two patient populations. However, given the number of studies analyzed, it is premature to draw any definitive conclusions beyond that these are interesting data points from which a hypothesis can be generated. All of the observed data for Qysmia fit the model well, see FIG. 8. The decay coefficient was estimated to be 0.08±0.01. The decay coefficient was very consistent across all the studies and doses. Given these were registration studies, the larger sample sizes may have allowed for better resolution. The observed data for sibutramine also fit the model well. Since there were more studies, it was impractical to show them all in a graph. The decay coefficient was estimated to be 0.14±0.04. Sibutramine offered the widest number of conditions studied, and as expected, there was more variability in the decay coefficient. That said, no correlation was found between the decay coefficient and dosage, and a χ² test of the normalized residuals suggested the variation was due to random noise (p<0.05).

TABLE 5
summarizes the values estimated for the model
parameters for all weight loss agents reviewed

| | | | ΔWt. | Decay |
|---|---|---|---|---|
| Weight loss agent | Dosage | | Loss_{MAX} | Coeff. (−b) |
| Sibutramine | 1 | mg | 3.7 kg | 0.14 ± 0.04 |
| (n = 8) | 5 | mg | 4.5 kg | |
| | 10 | mg | 8.4 kg | |
| | 15 | mg | 7.3 kg | |
| | 20 | mg | 8.5 kg | |
| | 30 | mg | 9.1 kg | |
| Qysmia | 3.75/23 | mg | 9.8 kg | 0.08 ± 0.01 |
| | 15/92 | mg | 14.3 kg | |
| Orlistat | 120 mg (diabetics) | | 4.3 kg | 0.11 ± 0.02 |
| | 120 | mg | 7.1 kg | |
| C. sinensis | 500-700 | mg | 2.0 kg | 0.11 |
| alginate | 45 | g | 9.6 kg | 0.09 |

There are several observations worth noting as areas of interest for further study and validation.

Agreement Between the Observed Data and the Model Supports the Adaptation Argument.

If there was nothing acting to counter the weight loss agent, the response curve would be linear. The fact that regardless of agent being used, each follows the same decay curve, suggests that there is some biological adaptation occurring which ultimately negates the effect.

ΔWt. Loss_{MAX} May be Correlated to Both the Strength of the Agent & Patient Population

The model values for ΔWt. Loss_{MAX} are in line with the accepted beliefs on effectiveness of the agents. For example, sibutramine appears directionally stronger than orlistat, and both are stronger than green tea alone. The ΔWt. Loss_{MAX} also appears to agree nicely with the effect size estimates in section 3.1.2. It may be interesting to see how well this model describes observational data outside of the studies used to develop the model. Another area to explore in more detail is the potential link between ΔWt. Loss_{MAX} and patient populations. Understanding if there is a link between patient co-morbidity and ΔWt. Loss_{MAX} is important since ΔWt. Loss_{MAX} sets the boundary conditions for the model.

Decay Coefficient May be Linked to the Weight Loss Agent Used

Given the wide range of agents, it was somewhat surprising to see that the decay coefficient varied over a narrow range 0.08-0.14. These small differences in the decay coefficient can be significant. For example, the difference in ½ life (time to which ½ the effect is gone) between 0.08 and 0.14 is about 3-4 weeks. Said differently, when ½ of the effect of higher coefficient weight loss agent is gone, only a ⅓ of the lower one is. While this would be important if the total effect sizes were large, in this case, however, the maximum absolute difference would likely be in the order of 1-2 kg, making it difficult to detect. Therefore, more work is needed to determine whether the decay coefficient is a fundamental constant or if it is truly a function of agent used. So far, the data seem to suggest that it may be a function of the agent used. It is reasonable to expect that the body might more rapidly adapt to one mechanism over another, or adapt more slowly to a combination of weight loss agents. However, this remains to be proven. The fact that the effect of certain weight loss agents may decay faster than others may play a role in setting the duration of a given cycle. Since the decay characteristics of many of the weight loss agents are unknown, this could prove to be a complicating factor. However, given the absolute difference due to this effect is relatively small, this effect may be negligible.

Key Implications

The fundamental hypothesis being tested is whether the decay curve is reset each time a new cycle starts. Given the seemingly fundamental relationship between the observed data and the decay model, it is logical to assume that each cycle may also exhibit this relationship. The underlying theory behind cycling is that it resets the decay curve each time the treatment is switched, thereby creating a series of short decay curves. Since the rate of change is greatest during the earliest time period, this should maximize the rate of weight loss so that it is superior to only one step. Mathematically, these are the two functions being compared:

Weight (t)=ΔWt_{MAX}e^{-bt}+(Weightₜ₌₀−ΔWt_{MAX}) Single Cycle:

Weight (t)=ΔWt_{MAX-cycle 1}e^{-bt}+(Weightₜ₌₀−ΔWt_{MAX-cycle 1})|ₜ₌₀^{t=4}+ΔWt_{MAX-cycle 2}e^{-b(t-4)}+(Weightₜ₌₄−ΔWt_{MAX-cycle 2})|ₜ₌₄^{t=8}+ΔWt_{MAX-cycle 3}e^{-b(t-8)}+(Weightₜ₌₈−ΔWt_{MAX-cycle 3})|ₜ₌₈^{t=12}+(n) Multiple Cycles:

Where n is equal to the number of cycles.

Study Objectives

Null Hypothesis 1 (H₀₋₁):

Treatment regimen is not shown to demonstrate a consistent rate of weight loss, that is, the rate as measured in pounds lost per week is significantly less in cycles 2 and 3 than cycle 1.

Null hypothesis 2 (H₀₋₂):

Treatment regimen is not shown to increase the time to weight loss plateau. The rate of weight loss hits a plateau phase by week 12. Since the decay curves are well established, a positive control is not necessary. In addition, since the effect is being compared against itself, there is no need for a placebo control in the initial study.

Study Design

Primary Endpoints

TABLE 6
summarizes the primary endpoints.

| Claims | Primary Endpoints |
|---|---|
| Product is shown to demonstrate | Weekly weight loss. |
| a consistent rate of weight loss. | Rate of weight loss per week. |
| | Rate of weight loss per month. |
| | Change in rate of weight loss from |
| | Cycle 1 to Cycle 2. |
| | Change in rate of weight loss from |
| | Cycle 2 to Cycle 3. |
| | Change in rate of weight loss from |
| | Cycle 1 to Cycle 3. |
| Product is shown to increase the | Time (in weeks) to weight loss plateau. |
| time to weight loss plateau. | |
| Product is shown to be safe and | Adverse Events. |
| well tolerated | |

Design

12 week open label study of 5 overweight, but otherwise relatively healthy subjects, ages 20 to 70, 2 Females and 3 Males. Weight were documented and recorded twice per week using photographs of the scales. No changes in diet or exercise were made.

Results

There was No Significant Decrease in Weight Loss Between the Cycles

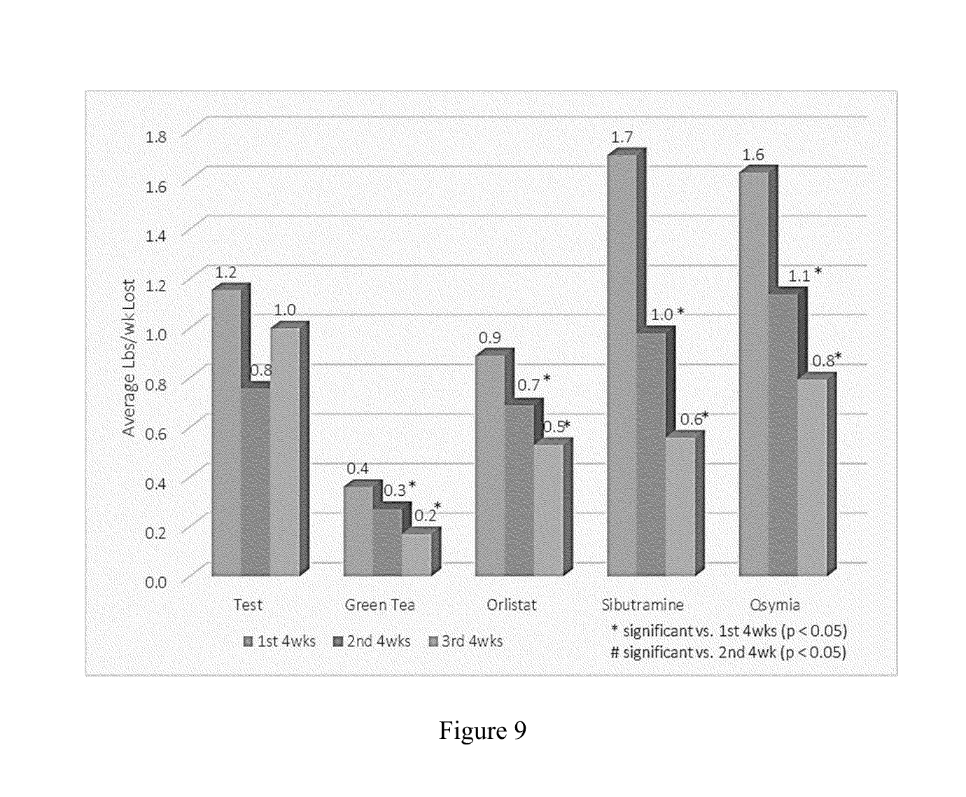

Unlike all the other treatments modeled, there was no significant change in the rate of weight loss per week between the three cycles. Cycle 1 (0-4-wks) was not significantly different from Cycle 2 (4-8 wks) or Cycle 3 (8-12 wks). Cycle 2 was not significantly different from Cycle 3. The results are summarized in FIG. 9.

There was No Indication of a Weight Loss Plateau at 12 Weeks

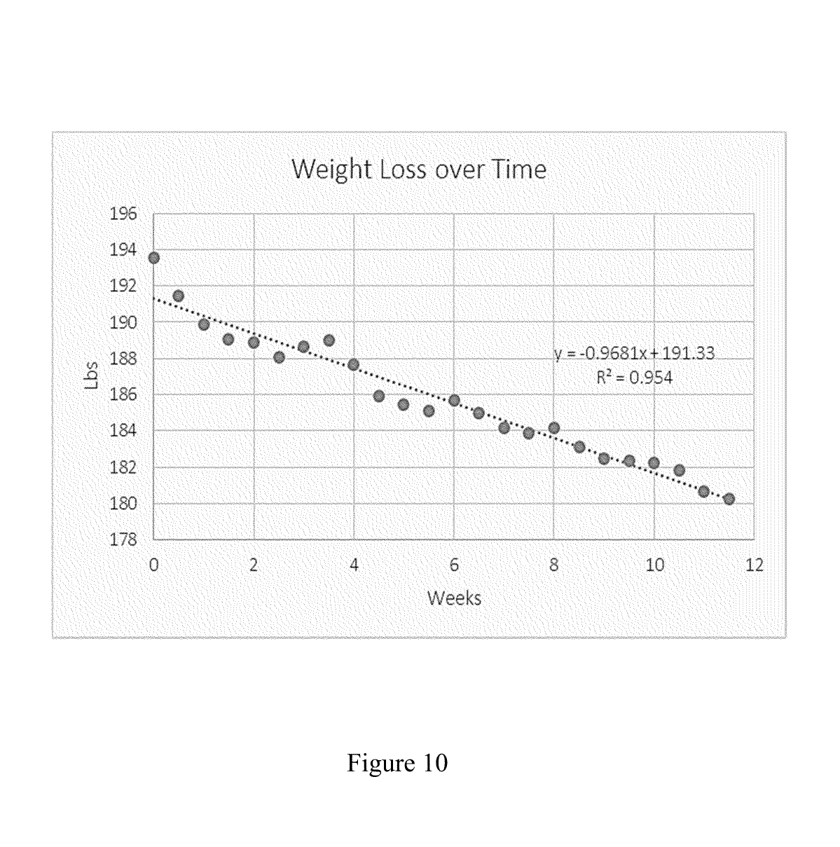

Unlike what has been show with other treatments, there was a linear relationship between weight loss and time, which did not decrease over time, as shown in FIG. 10. The same was true for BMI.

Average Weight Loss was 13.7 Pounds and was Significantly Lower than Baseline

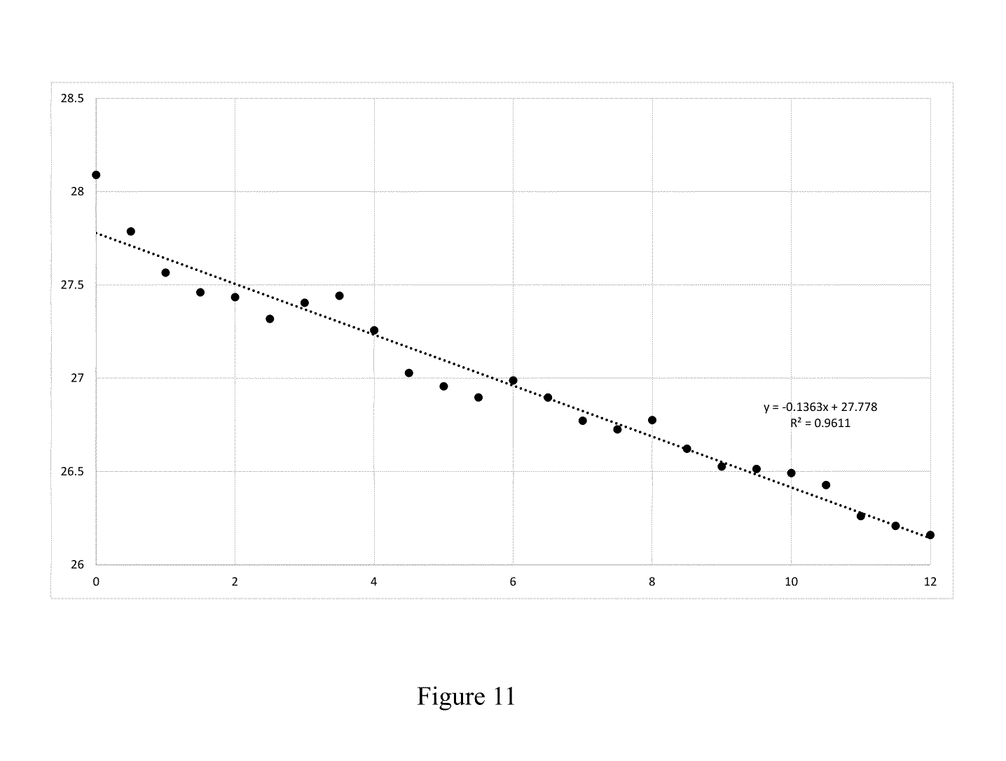

The average weight loss was 13.7 pounds and was statistically lower than baseline (p<<0.01). The average BMI change was 1.9, and this also was highly significant vs. baseline (p<0.01), see FIG. 11.

### Example 5

### Estimates of Weight Loss by Weight Loss Agent

A rough point estimate of the effect size by weight loss agent was developed using data from published studies. These data should be used with caution since there is significant variability in design quality, patient populations, dosing, and study design. However, it is possible to use these data to develop hypotheses, which then can be demonstrated in the clinic.

TABLE 7
Summary of anticipated weight loss of particular weight loss agents.

| Weight loss agent | Dosage | | Kcal/d | Kg./wk | ΔWt. Loss_{MAX} | |
|---|---|---|---|---|---|---|
| Camellia sinensis | 500-700 | mg/day | 63.5-89 | 0.15-0.2 | 2.0 | kg |
| Capsicum sp. | 15-30 | mg/day | 50-75 | | | |
| (pungent) | | | | | | |
| Garcinia sp. | 1,500-2,000 | mg/day | | 0.1-0.2 | 1.0 kg | |
| | | | | | (<1500 mg) | |
| | | | | | 3.0 kg (3000 mg) | |
| Citrus aurantium | 50 | mg/day | 40 | | | |
| Phaseolus vulgaris | 2,000-3,000 | amu² | | 0.2-0.25 | 3.0-3.5 | kg |
| Caffeine | 500-700 | mg/day | 50-75 | | | |
| yohimbine | 20 | mg/day | | 0-0.16 | 0-1.5 | kg |
| Zotrim ® | Commercial | | 144 | 0.2-0.4 | 2.5-3.0 | kg |
| | formula | | | | | |
| 5- | 50-900 | mg oral | 400 | 0.2 | 1.5-2.5 | kg |
| hydroxytryptophan | | | | | | |
| Coffea canefora | 300-500 | mg/day | | 0.3 +/− 0.1 | 2.5 | kg |
| Slendesta ® | 15-30 | mg | | 0.1 | 0.7 | kg |
| Coleus forskholii | 500 | mg | | 0.1-0.2 | 1.5 | kg |
| Cissus | 300 | mg | | 0.6 +/− 0.2³ | 6.0 | kg³ |
| quadrangularis | | | | | | |
| Irvingia gabonensis | 300 | mg | | 1.0 +/− 0.1 | 10.0 | kg |

| Phospholean ® | Commercial | | Decreased | n.s. |
|---|---|---|---|---|
| | formula | | binge eating | |
| Ecklonia cava | 144 | mg | | 0.1 (1 study) |

| | | | | |
|---|---|---|---|---|
| Sinetrol ® | Commercial | 0-0.2 (trend) | 2.0 | kg (trend) |
| | formula | | | |

| Glycyrrhiza sp. | | | 0.1 (1 study) |
|---|---|---|---|
| St. John's Wort | 27 mg | | n.s. (1 study) |
| | (hypericum) | | |
| Olea Europaea | 150 | mg | too variable |
| Rhodiola rosea | 300-600 | mg | ↓ fatigue |
| Crocus sativus | 176.5 | mg (stigma) | 0.1 (1 study) |

The ΔWt. Loss_{MAX} for Garcinia sp. from 1.0 kg to 3.0 kg assumes the dose response curve model is valid, which has not been proven. The dosage of Phaseolus vulgaris is measured in α-amylase blocking units. Coffea canefora is administered as Syetol®, a commercial preparation most commonly used in the studies (400 mg). Crocus sativus dosages for depression are much lower 30 mg and have been shown for extracts of both stigma & leaf.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges, which can be subsequently broken down into subranges as discussed above. Finally, as will be understood by one skilled in the art, a range includes each individual member. Thus, for example, a group having 1-3 substituents refers to groups having 1, 2, or 3 substituents. Similarly, a group having 1-5 substituents refers to groups having 1, 2, 3, 4, or 5 substituents, and so forth.

What is claimed:

1. A method of effecting weight loss in a subject, the method comprising:
defining at least two treatment phases, wherein each of the at least two treatment phases has a pre-determined duration; and
administering at least one weight loss agent during each treatment phase;
wherein the at least one weight loss agent administered during each treatment phase is selected from a different therapeutic class than the at least one weight loss agent administered during the preceding or proceeding treatment phase.

2. The method of claim 1, wherein the therapeutic class is selected from a neurotransmitter modulator, a hormonal satiety or a hunger signaling modulator, a β-adrenergic receptor modulator, an α2-adrenergic modulator, an adenosine receptor modulator, a cannabinoid receptor modulator, a protein-tyrosine phosphatase 1B modulator, a catechol O-methyltransferase modulator, an AMPk modulator, a phosphodiesterase modulator, a non-guanosine nucleotide mediated adenylate cyclase modulator, a glucose metabolism modulator, a carbohydrate metabolism modulator, a lipid metabolism modulator and an agent with an unknown mechanism of action.

3. The method of claim 1, wherein each of the at least two treatment phases has a pre-determined duration of about 1 week to about 20 weeks.

4. The method of claim 1, wherein each of the at least two treatment phases has a pre-determined duration of about 4 weeks.

5. The method of claim 1, wherein the at least two weight loss agents are administered during each treatment phase, wherein the at least one weight loss agent administered during each treatment phase is selected from a different therapeutic class than the at least one weight loss agent administered during the preceding or proceeding treatment phase.

6. A method of prolonging the effect of a weight loss agent in a subject, the method comprising:
defining at least two treatment phases, wherein each of the at least two treatment phases has a pre-determined duration;
administering at least one weight loss agent during each treatment phase;
wherein the at least one weight loss agent administered during each treatment phase is selected from a different therapeutic class than the at least one weight loss agent administered during the preceding or proceeding treatment phase.

7. The method of claim 6, wherein the therapeutic class is selected from a neurotransmitter modulator, a hormonal satiety or a hunger signaling modulator, a β-adrenergic receptor modulator, an α2-adrenergic modulator, an adenosine receptor modulator, a cannabinoid receptor modulator, a protein-tyrosine phosphatase 1B modulator, a catechol O-methyltransferase modulator, an AMPk modulator, a phosphodiesterase modulator, a non-guanosine nucleotide mediated adenylate cyclase modulator, a glucose metabolism modulator, a carbohydrate metabolism modulator, a lipid metabolism modulator and an agent with an unknown mechanism of action.

8. The method of claim 6, wherein each of the at least two treatment phases has a pre-determined duration of about 1 week to about 20 weeks.

9. The method of claim 6, wherein each of the at least two treatment phases has a pre-determined duration of about 4 weeks.

10. The method of claim 6, wherein the at least two weight loss agents are administered during each treatment phase, wherein the at least one weight loss agent administered during each treatment phase is selected from a different therapeutic class than the at least one weight loss agent administered during the preceding or proceeding treatment phase.

11. A kit for effecting weight loss in a subject, the kit comprising:
at least two weight loss agents; and
instructions for defining at least two treatment phases and administering at least one weight loss agent during each treatment phase, wherein each of the at least two treatment phases has a pre-determined duration;
wherein the at least one weight loss agent administered during each treatment phase is selected from a different therapeutic class than the at least one weight loss agent administered during the preceding or proceeding treatment phase.

12. The kit of claim 11, wherein the therapeutic class is selected from a neurotransmitter modulator, a hormonal satiety or a hunger signaling modulator, a β-adrenergic receptor modulator, an α2-adrenergic modulator, an adenosine receptor modulator, a cannabinoid receptor modulator, a protein-tyrosine phosphatase 1B modulator, a catechol O-methyltransferase modulator, an AMPk modulator, a phosphodiesterase modulator, a non-guanosine nucleotide mediated adenylate cyclase modulator, a glucose metabolism modulator, a carbohydrate metabolism modulator, a lipid metabolism modulator and an agent with an unknown mechanism of action.

13. The kit of claim 11, wherein each of the at least two treatment phases has a pre-determined duration of about 1 week to about 20 weeks.

14. The kit of claim 11, wherein each of the at least two treatment phases has a pre-determined duration of about 4 weeks.

15. The kit of claim 11, wherein the at least two weight loss agents are administered during each treatment phase, wherein the at least one weight loss agent administered during each treatment phase is selected from a different therapeutic class than the at least one weight loss agent administered during the preceding or proceeding treatment phase.
